(19) United States
(12) Patent Application Publication
(10) Pub. No.: US 2024/0058368 A1
(43) Pub. Date: Feb. 22, 2024

(54) Respirable Polynucleotide Powder Formulations for Inhalation

(71) Applicant: Civitas Therapectics, Inc., Pearl River, NY (US)

(72) Inventors: Adam Hartman, Medford, MA (US); Fahad Gilani, Wayland, MA (US); Kyle LaVigne, Ashland, MA (US); Michael M. Lipp, Framingham, MA (US)

(21) Appl. No.: 18/121,097

(22) Filed: Mar. 14, 2023

Related U.S. Application Data
(63) Continuation of application No. 17/865,774, filed on Jul. 15, 2022, now abandoned.
Application No. 17/865,774 is a continuation of application No. 17/546,214, filed on Dec. 9, 2021, now abandoned.
Application No. 17/546,214 is a continuation of application No. 17/313,360, filed on May 6, 2021, now abandoned.
Application No. 17/313,360 is a continuation of application No. PCT/US19/61237, filed on Nov. 13, 2019.
(60) Provisional application No. 62/760,232, filed on Nov. 13, 2018.

(51) Int. Cl. A61K 31/7105; A61K 9/16

(52) U.S. Cl. CPC A61K 31/7105; A61K 9/1623; A61K 9/1611

(57) ABSTRACT

The invention provides respirable dry powder particle formulations comprising polynucleotides preferably prepared by spray drying for delivery to the pulmonary system via inhalation. Preferably, the polynucleotide is RNA.

### RELATED APPLICATIONS

This application is a continuation of U.S. application Ser. No. 17/865,774, filed on Jul. 15, 2022, which is a continuation of U.S. application Ser. No. 17/546,214, filed on Dec. 9, 2021, now abandoned, which is a continuation of U.S. application Ser. No. 17/313,360, filed on May 6, 2021, now abandoned, which is a continuation of International Application No. PCT/US19/61237, which designated the United States and was filed on Nov. 13, 2019, published in English, which claims the benefit of U.S. Provisional Application No. 62/760,232, filed Nov. 13, 2018. The entire teachings of the above applications are incorporated herein by reference.

### BACKGROUND OF THE INVENTION

The development of respirable polynucleotide-containing powder particle formulations, for example, RNA dry powder particle formulations with therapeutically relevant levels of RNA for delivery to the pulmonary system via inhalation, would provide a noninvasive and targeted approach in the treatment of diseases, as well as an improvement over nebulizers and pressured metered dose inhalers.

### SUMMARY OF THE INVENTION

The invention provides respirable dry powder particle formulations comprising polynucleotides prepared by spray drying for delivery to the pulmonary system via inhalation. Preferably, the polynucleotide is RNA. Preferably, the invention provides respirable polynucleotide dry powder particle formulations prepared by spray drying for delivery to the pulmonary system via inhalation. The invention comprises formulations and parameters used in spray drying polynucleotides, for example, an RNA-containing powder such as an mRNA-containing powder, with suitable chemical, physical and aerosol properties for pulmonary delivery, preferably delivery using a dry powder inhaler that is preferably patient activated. See for example the ARCUS® platform, for inhaled medicines described at www.acorda.com.

Preferably, the respirable, polynucleotide dry powder particle formulation for pulmonary delivery comprises:
i) at least about 1% polynucleotide (e.g., RNA) by weight of the particle;
ii) at least about 10% DPPC by weight of the particle;
iii) optionally, at least about 1% each of one or more of arginine, leucine,
isoleucine, or valine by weight of the particle, wherein when two or more amino acids are selected, each amino acid has either the same or different weight percentage as the other selected amino acid(s);
iv) optionally, at least about 1% NaCl by weight of the particle;
v) optionally, at least about 10% Tris by weight of the particle;
vi) optionally, at least about 2% EDTA by weight of the particle;
vii) optionally, at least about 40% trehalose by weight of the particle;
viii) optionally, at least about 2% PEI by weight of the particle; and
ix) optionally, at least about 40% lactose by weight of the particle.
wherein all components of the RNA dry powder amount to 100 weight percent.

Preferably the formulation comprises about 1% to about 80%, and preferably about 1% to 70%, such as about 1%, 10%, 25%, 50%, or about 60% by weight of a polynucleotide. Preferably the polynucleotide is RNA. The term “RNA” is intended to cover all RNA and modified RNA in all forms and from all sources. Preferably the RNA is mRNA. The mRNA can be capped or uncapped mRNA. Preferably the mRNA is capped RNA. In the Examples using yeast RNA, the yeast RNA is as a placeholder for mRNA.

Preferably the polynucleotide can be at least about 10, 15, 16, 17, 18, 19, 20, 25, 30, 35, 40, 45, 50, 55, 60, 65, 70, 75, 80, 85, 90, 100, 125, 150, 200 or more nucleic acids in length. Preferably, the RNA may be from about 10 to about 12,000 nucleic acids or more in length. Preferably, the RNA may be from about 20 to about 12,000 or more nucleic acids in length. Preferably, the RNA may be from about 50 to about 12,000 or more nucleic acids in length. Preferably the RNA may be from about 100 to about 12,000 or more nucleic acids in length. Preferably the RNA can be from about 20 to about 12,000 monomers in length, or about 20 to about 10,000 monomers, or about 20 to 8,000 monomers, or about 20 to 6000 monomers, or about 20 to about 5000 monomers, or about 20 to about 4000 monomers, or about 20 to about 3600 monomers, or about 20 to about 3200 monomers, or about 20 to about 3000 monomers, or about 20 to about 2800 monomers, or about 20 to about 2600 monomers, or about 20 to 2400 monomers, or about 20 to 2200 monomers, or about 50 to 3200 monomers, or about 50 to 3000 monomers, or about 50 to 2600 monomers.

Preferably the formulation comprises about 10% to about 30%, and preferably about 15% to 25%, such as about 15%, 18%, 20%, 22%, or about 25% by weight of DPPC.

The invention provides a respirable, polynucleotide (e.g., RNA) dry powder formulation that can optionally comprise at about 1% and preferably from about 1% to about 20%, preferably from about 1% to about 10% and preferably from about 1% to about to about 5% of a cationic excipient. The cationic excipient can be at least one positively charged amino acid or polyethylenimine (PEI).

Preferably the formulation optionally comprises about 1% to about 90%, and preferably about 5% to 85%, such as about 5%, 20%, 30%, 40%, 50%, 55%, 70%, 74%, 79%, 80%, or about 85% by weight of one or more of arginine, leucine, isoleucine, or valine, wherein when two or more amino acids are selected, each amino acid has either the same or different weight percentage as the other selected amino acid(s).

Preferably the formulation optionally comprises about 1% to about 5%, and preferably about 1% to 3%, such as about 1%, 2%, or about 3% by weight of NaCl.

Preferably the formulation optionally comprises about 1% to about 25%, and preferably about 5% to 20%, such as about 5%, 10%, 15%, or about 20% by weight of Tris.

Preferably the formulation optionally comprises about 1% to about 10%, and preferably about 2% to 8%, such as about 2%, 3%, 4%, 5%, 6%, or about 7% by weight of EDTA.

Preferably the formulation further optionally comprises about 40% to about 90%, and preferably about 45% to 85%, such as about 45%, 50%, 52%, 53%, 54%, 55%, 60%, 65%, 70%, 72%, 74%, 75%, 77%, 79%, 80%, or about 82% by weight of trehalose.

Preferably the formulation further optionally comprises about 40% to about 90%, and preferably about 45% to 85%, such as about 45%, 50%, 52%, 53%, 54%, 55%, 60%, 65%, 70%, 72%, 74%, 75%, 77%, 79%, 80%, or about 82% by weight of lactose.

Preferably the formulation optionally comprises about 1% to about 10%, and preferably about 1% to 8%, such as about 1%, 2%, 3%, 4%, 5%, 6%, or about 7% by weight of PEI.

Preferably, the formulation is selected from one of the following formulations listed in Table 1.

| Number | Formulation |
|---|---|
| 1 | RNA:Arginine:DPPC:NaCl (10:70:18:2) |
| 2 | RNA:Trehalose:DPPC:Tris:EDTA (10:53:18:15:4) |
| 3 | RNA:Trehalose:DPPC:Tris:EDTA (10:52:18:15:5) |
| 4 | RNA:Leucine:DPPC:NaCl (1:79:18:2) |
| 5 | RNA:Trehalose:DPPC:NaCl (1:79:18:2) |
| 6 | RNA:Trehalose:DPPC:PEI:NaCl (1:74:18:5:2) |
| 8 | RNA:Trehalose:DPPC:PEI:NaCl (10:65:18:5:2) |
| 9 | RNA:Trehalose:DPPC:NaCl (25:55:18:2) |
| 11 | RNA:Isoleucine:DPPC:NaCl (50:30:18:2) |
| 12 | RNA:Valine:DPPC:Arginine:NaCl (25:50:18:5:2) |
| 14 | RNA:Isoleucine:DPPC:NaCl (25:55:18:2) |
| 16 | RNA:Valine:DPPC:NaCl (25:55:18:2) |
| 17 | RNA:Leucine:DPPC:NaCl (25:55:18:2) |
| 18 | RNA:Leucine:DPPC:Arginine:NaCl (25:50:18:5:2) |
| 22 | RNA:Lactose:DPPC:NaCl (25:55:18:2) |
| 23 | RNA:SD-30:DPPC:NaCl (25:55:18:2) |
| 24 | RNA:Leucine:DPPC:Arginine:NaCl (25:50:18:5:2) |
| 25 | RNA:Trehalose:DPPC:NaCl (25:55:18:2) |
| 26 | RNA:Valine:DPPC:Arginine:NaCl (25:50:18:5:2) |
| 27 | RNA:Trehalose:DPPC:NaCl (1:79:18:2) |
| 29 | Uncapped mRNA:Trehalose:DPPC:NaCl (1:79:18:2) |
| 30 | RNA:Trehalose:DPPC:NaCl (25:55:18:2) |
| 34 | RNA:Leucine:DPPC:NaCl (25:55:18:2) |
| 35 | Uncapped mRNA:Leucine:DPPC:NaCl (1:79:18:2) |
| 36 | RNA:Lactose:DPPC:NaCl (25:55:18:2) |
| 37 | Uncapped mRNA:Lactose:DPPC:NaCl (1:79:18:2) |
| 39 | RNA:Leucine:DPPC:NaCl (1:79:18:2) |
| 40 | RNA:Lactose:DPPC:NaCl (1:79:18:2) |
| 44 | capped mRNA:Lactose:DPPC:NaCl (1:79:18:2) |
| 45 | capped mRNA:Lactose:DPPC:NaCl (10:70:18:2) |

Weight percent is intended to reflect the total amount of solids, lipids, and/or excipients in the dry particles without regard to residual water, solvent or impurities. Preferably, all the components of the dry particles amount to 100 wt %.

### BRIEF DESCRIPTION OF THE DRAWINGS

FIG. 1. Illustrates the spray dryer setup used for development of RNA powders.

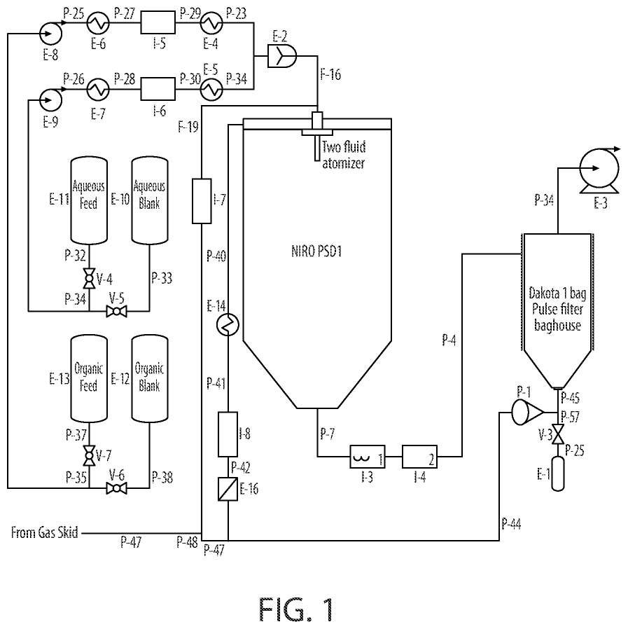

### DETAILED DESCRIPTION OF THE INVENTION

The terms “a”, “an” and “the” as used herein are defined to mean “one or more” and include the plural unless the context is inappropriate.

The term “comprising” as used herein which is synonymous with “including,” “containing,” or “characterized by,” is inclusive or open-ended and does not exclude additional, unrecited elements of a composition or method steps. The term “consisting of” excludes any element, step, or ingredient that is not otherwise specified. The term “consisting essentially of” limits the scope of a composition or method to the specified materials or steps and those that do not materially affect the basic and novel characteristic(s) of the specified composition or method.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or 10%, or more of the numerical value(s) being modified. In addition, the phrase “about ‘x’ to ‘y’” includes “about ‘x’ to about ‘y’”.

The term “dry powder” as used herein refers to a composition that contains finely dispersed respirable dry particles that are capable of being dispersed in an inhalation device and subsequently inhaled by a subject. Such dry powder or dry particle may contain up to about 15% water or other solvent, preferably up to about 10% water or other solvent, or preferably be substantially free of water or other solvent, or preferably be anhydrous.

The terms “dry particles” and “particle” as used herein refers to respirable particles that may contain up to about 15% water or other solvent, preferably up to 10% water or other solvent or preferably be substantially free of water or other solvent, or preferably be anhydrous.

The term “respirable” as used herein refers to dry particles or dry powders that are suitable for delivery to the respiratory tract (e.g., pulmonary delivery) in a subject by inhalation. Respirable dry powders or dry particles have a mass median aerodynamic diameter (MMAD) of less than about 10 microns, preferably about 5 microns and more preferably about 3 microns or less. The “mass median aerodynamic diameter” (MMAD) is also referred to herein as “aerodynamic diameter”. Experimentally, aerodynamic diameter can be determined by employing a gravitational settling method, whereby the time for an ensemble of powder particles to settle a certain distance is used to infer directly the aerodynamic diameter of the particles. An indirect method for measuring the mass median aerodynamic diameter (MMAD) is the multi-stage liquid impinger (MSLI). The aerodynamic diameter, der, can be calculated from the equation:

dₐₑᵣ=d_{g}√ρₜₐₚ
where d_{g} is the geometric diameter, for example the MMGD, and ρ is the powder density.

As used herein, the terms “administration” or “administering” of respirable dry particles refers to introducing respirable dry particles to the respiratory tract of a subject.

As used herein, the term “respiratory tract” includes the upper respiratory tract (e.g., nasal passages, nasal cavity, throat, pharynx), respiratory airways (e.g., larynx, trachea, bronchi, bronchioles) and lungs (e.g., respiratory bronchioles, alveolar ducts, alveolar sacs, alveoli). The deep lung, or alveoli, are typically the desired target of inhaled therapeutic formulations for systemic drug delivery. In one embodiment of the invention, most of the mass of particles deposit in the deep lung or alveoli. In another embodiment of the invention, delivery is primarily to the central airways. In other embodiments, delivery is to the upper airways.

“Pulmonary delivery,” as that term is used herein refers to delivery to the respiratory tract. Pulmonary delivery includes inhalation by a patient that is capable of independent inhalation or inhalation via a ventilation system such as a mechanical ventilation (MV) system or a non-invasive mechanical ventilation system (NIMV) such as via a continuous positive airway pressure (CPAP) system.

The term “gPSD₅₀” as used herein refers to the average geometric particle size diameter, wherein 50% of the particles have a diameter larger than the listed value, and 50% of the particles have a diameter smaller than the listed value.

The terms “FPF (<5.6),” “FPF (<5.6 microns),” “FPF<5.6 μm”, and “fine particle fraction of less than 5.6 microns” as used herein, refer to the fraction of a sample of dry particles that have an aerodynamic diameter of less than 5.6 microns. For example, a two- or three-stage collapsed ACI can be used to measure FPF<5.6 microns. The two-stage collapsed ACI consists of only the top stage (S0) and the filter stage of the eight-stage ACI and allows for the collection of two separate powder fractions. Specifically, a two-stage collapsed ACI is calibrated so that the fraction of powder that is collected on S0 is composed of non-respirable dry particles that have an aerodynamic diameter of greater than 5.6 microns. The fraction of powder passing S0 and depositing on the filter stage is thus composed of respirable dry particles having an aerodynamic diameter of less than 5.6 microns. The airflow at such a calibration is approximately 60 L/min. This parameter may also be identified as “FPF TD(<5.6),” where TD means total dose. A similar measurement can be conducted using an eight-stage ACI. The eight-stage ACI cutoffs are different at the standard 60 L/min flowrate, but the FPF TD(<5.6) can be extrapolated from the eight-stage complete data set. The eight-stage ACI result can also be calculated by the USP method of using the dose collected in the ACI instead of what was in the capsule to determine FPF.

The terms “FPF (<3.4),” “FPF (<3.4 microns),” “FPF<3.4 μm”, and “fine particle fraction of less than 3.4 microns” as used herein, refer to the fraction of a mass of respirable dry particles that have an aerodynamic diameter of less than 3.4 microns. For example, three-stage collapsed ACI can be used to measure both FPF<5.6 microns and <3.4 microns. The three-stage collapsed ACI consists of collection stage S0, S2 and the filter stage and provides fractions of powder of an aerodynamic diameter greater than 5.6 microns, less than 5.6 microns and less than 3.4 microns. This parameter may also be identified as “FPF TD(<3.4),” where TD means total dose. A similar measurement can be conducted using an eight-stage ACI. The eight-stage ACI result can also be calculated by the USP method of using the dose collected in the ACI instead of what was in the capsule to determine FPF. Other cutoff values for FPF (i.e., <5.0 microns, etc.) can be utilized in a similar manner either via utilizing different stage configurations for the ACI or else extrapolating from the results obtained for a specific set of stages and cutoff diameters.

Preferred “excipients” as that term is used herein are those excipients that can be taken into the lungs. Such excipients can be generally regarded as safe (GRAS) by the U.S. Food and Drug Administration. Such excipients include water.

The term “patient” or “subject” as used interchangeably herein are individuals to whom the compositions of the invention may be administered. Examples of such individuals include elderly humans, adult humans and pediatric humans. Pediatric humans include individuals aged from birth up to 18 years of age. Pediatric aged children may also include the following subgroups including but not limited to, neonates comprising newborn individuals up to about 28 days of age or 1 month of age; infants comprising individuals aged from the neonatal period up to 12 months of age; toddlers comprising individuals of ages 1-3 years old; preschool children comprising individuals of ages 3-5 years old, school-aged children comprising individuals of ages 6 to 10 years old and adolescents comprising individuals of ages 11-14 years. Pediatric children may also be referred to having the following age ranges of about 6 to about 11 years of age, about 12 to about 17 years of age, or about 6 to about 17 years of age. Premature human children (preemies) include individuals who are less than about 37 weeks of gestational age. Elderly humans may be at least 50 years of age, such as at least 65 years of age, such as 70 years or more.

The invention further provides for packaging respirable dry powders in the presence of a desiccant, such as silica gel desiccant; a zeolite; an alumina; a bauxite; anhydrous calcium sulphate; water-absorbing clay; a molecular sieve; and any mixtures thereof. The desiccant can be packaged in a sachet, pouch or pack and placed in the packaging with the capsules. The desiccant can also be integrated into the packaging itself, such as by coating, absorption or adsorption. For example, a film sealing a blister pack containing a capsule can comprise the desiccant, such as by integration, absorption or adsorption. The packaging can be further sealed with a moisture barrier such as a bottle or blister with a foil seal.

The desiccant may be added in an amount effective to reduce moisture within the packaging during storage and will depend on the size of the container and the internal exposure conditions to humidity. For example, 1 g of silica gel is sufficient to protect a package containing 4 Size 00 capsules (HPMC capsules). It is preferred that the humidity, or volatiles content, within the packaging is reduced to less than 5 wt %, preferably less than 3 wt %, such as less than 1 wt %. Preferably, the amount of desiccant is added to maintain or increase the FPF value after 2 weeks, such as 4 weeks, of storage at 40° C. and 75% relative humidity.

The term “Size 00 FPF<5.6 μm (%)” is intended to mean the percent of particles with an aerodynamic diameter <5.6 microns in the capsule tested. Preferably, the “Size 00 FPF<5.6 μm (%)” is at least about 50%, preferably >55%, >60%, >65%, >70%, >75%, >80%, >85%, >90%, >95%, or 100%. Preferably, the “Size 00 FPF<5.6 μm (%)” after storage is at least about 50%, preferably >55%, >60%, >65%, >70%, >75%, >80%, >85%, >90°/%, >95%, or 100%.

The term “Size 00 FPF<3.4 μm (%)” is intended to mean the percent of particles with an aerodynamic diameter <3.4 microns in the capsule tested. Preferably, the “Size 00 FPF<3.4 μm (%)” is at least about 35%, preferably at least 50%, >55%, >60%, >65%, >70%, >75%, >80%, >85%, >90%, >95%, or 100%. Preferably, the “Size 00 FPF<3.4 μm (%)” after storage is at least about 50%, preferably >55%, >60%, >65%, >70%, >75%, >80%, >85%, >90%, >95%, or 100%.

The term “nucleic acid,” in its broadest sense, includes any compound and/or substance that comprise a polymer of nucleotides. These polymers are often referred to as polynucleotides. Exemplary nucleic acids or polynucleotides described herein include, but are not limited to, ribonucleic acids (RNAs), short interfering RNA (siRNA), micro-RNA, deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

As used herein, the term “polynucleotide” is generally used to refer to a nucleic acid (e.g., DNA or RNA). When RNA, such as mRNA, is specifically being referred to, the term polyribonucleotide may be used. The terms polynucleotide, polyribonucleotide, nucleic acid, ribonucleic acid, DNA, RNA, mRNA, and the like include such molecules that may be comprised of standard or unmodified residues; nonstandard or modified residues (e.g., analogs); and mixtures of standard and nonstandard (e.g., analogs) residues. In certain embodiments a polynucleotide or a polyribonucleotide is a modified polynucleotide or a polyribonucleotide. In the context of the present disclosure, for each RNA (polyribonucleotide) sequence listed herein, the corresponding DNA (polydeoxyribonucleotide or polynucleotide) sequence is contemplated and vice versa. “Polynucleotide” may be used interchangeably with the “oligomer.”

As used herein, the term “messenger RNA” (mRNA) refers to any polynucleotide which encodes a protein or polypeptide of interest and which is capable of being translated to produce the encoded protein or polypeptide of interest in vitro, in vivo, in situ or ex vivo. “Protein” and “polypeptide” are used interchangeably herein and intended to include proteins and fragments thereof.

As used herein, the term “translation” is the process in which ribosomes create polypeptides. In translation, messenger RNA (mRNA) is decoded by transfer RNAS (tRNAs) in a ribosome complex to produce a specific amino acid chain, or polypeptide. The coding region of a polynucleotide sequence (DNA or RNA), also known as the coding sequence or CDS, is capable of being converted to a protein or a fragment thereof by the process of translation.

As used herein, the term “codon-optimized” means a natural (or purposefully designed variant of a natural) coding sequence which has been redesigned by choosing different codons without altering the encoded protein amino acid sequence. Codon optimized sequence can increase the protein expression levels (Gustafsson et al., Codon bias and heterologous protein expression. 2004, Trends Biotechnol 22: 346-53) of the encoded proteins amongst providing other advantages. Variables such as high codon adaptation index (CAI), LowU method, mRNA secondary structures, cis-regulatory sequences, GC content and many other similar variables have been shown to somewhat correlate with protein expression levels (Villalobos et al., Gene Designer: a synthetic biology tool for constructing artificial DNA segments. 2006, BMC Bioinformatics 7:285). High CAI (codon adaptation index) method picks a most frequently used synonymous codon for an entire protein coding sequence. The most frequently used codon for each amino acid is deduced from 74,218 protein-coding genes from a human genome. The Low U method targets only U-containing codons that can be replaced with a synonymous codon with fewer U moieties. If there are a few choices for the replacement, the more frequently used codon will be selected. The remaining codons in the sequence are not changed by the Low U method. This method may be used in conjunction with the disclosed mRNAs to design coding sequences that are to be synthesized with, for example, 5-methoxyuridine or N¹-methylpseudouridine.

As used herein, “modified” refers to a change in the state or structure of a molecule disclosed herein. The molecule may be changed in many ways including chemically, structurally or functionally. Preferably a polynucleotide or polypeptide of the disclosure are modified as compared to the native form of the polynucleotide or polypeptide or as compared to a reference polypeptide sequence or polynucleotide sequence. For example, mRNA disclosed herein may be modified by codon optimization, or by the insertion of non-natural nucleosides or nucleotides. Polypeptides may be modified, for example, by site specific amino acid deletions or substitutions to alter the properties of the polypeptide.

As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes. In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984), BLASTP, BLASTN, and FASTA Altschul, S. F. et al., J. Molec. Biol., 215, 403 (1990).

An “effective amount” of the mRNA sequence encoding an open reading frame (ORF) protein or a corresponding composition thereof can be that amount of mRNA that provides ORF protein production in a cell. Preferably protein production using an mRNA composition described herein is more efficient than a composition containing a corresponding wild type mRNA encoding an ORF protein. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell. When referring to an ORF protein described herein, an effective amount is that amount of ORF protein that overcomes an ORF protein deficiency in a cell. Where the mRNA is an mRNA vaccine, an effective amount includes an amount that induces immunity against an infection, such as influenza or respiratory syncytial virus, or cancer, or the like.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, cell or tissue thereof).

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound described herein. Substantial separation can include compositions containing at least about 50%, at least about 60%, at least about 70%, at least about 80%, at least about 90%, at least about 95%, at least about 97%, or at least about 99% by weight of the compound described herein, or salt thereof. Methods for isolating compounds and their salts are routine in the art.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “therapeutically effective amount” means an amount of an agent to be delivered (e.g., nucleic acid, protein or peptide, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, a “total daily dose” is an amount given or prescribed in a 24 hr period. It may be administered as a single unit dose.

As used herein, the term “treating” refers to partially or completely alleviating, ameliorating, improving, relieving, delaying onset of, inhibiting progression of, reducing severity of, and/or reducing incidence of one or more symptoms or features of a protein deficiency. Treatment may be administered to a subject who does not exhibit signs of said protein deficiency and/or to a subject who exhibits only early signs of the protein deficiency for the purpose of decreasing the risk of developing pathology associated with the disease, disorder, and/or condition.

As used herein, the terms “transfect” or “transfection” mean the intracellular introduction of a nucleic acid into a cell, or preferably into a target cell. The introduced nucleic acid may be stably or transiently maintained in the target cell. The term “transfection efficiency” refers to the relative amount of nucleic acid taken-up by the target cell which is subject to transfection. In practice, transfection efficiency is estimated by the amount of a reporter nucleic acid product expressed by the target cells following transfection. Preferred are compositions with high transfection efficacies and in particular those compositions that minimize adverse effects which are mediated by transfection of non-target cells and tissues.

As used herein, the term “target cell” refers to a cell or tissue to which a composition of the disclosure is to be directed or targeted. In some embodiments, the target cells are deficient in a protein or enzyme of interest. For example, where it is desired to deliver a nucleic acid to an epithelial or lung cell, the epithelial or lung cell represents the target cell. In some embodiments, the nucleic acids and compositions of the present disclosure transfect the target cells on a discriminatory basis (i.e., do not transfect non-target cells).

Following transfection of one or more target cells by the compositions and nucleic acids described herein, expression of the protein encoded by such nucleic acid may be preferably stimulated and the capability of such target cells to express the protein of interest is enhanced. For example, transfection of a target cell with a mRNA will allow expression of the modified protein product following translation of the nucleic acid. The nucleic acids of the compositions and/or methods provided herein preferably encode a product (e.g., a protein, enzyme, polypeptide, peptide, functional RNA, and/or antisense molecule), and preferably encode a product whose in vivo production is desired.

### Respirable Polynucleotide Dry Powder Particle Formulations

The invention provides respirable dry powder particle formulations comprising polynucleotides prepared by spray drying for delivery to the pulmonary system via inhalation. Preferably, the polynucleotide is RNA. Preferably, the invention provides respirable RNA dry powder particle formulations prepared by spray drying for delivery to the pulmonary system via inhalation. The invention provides respirable RNA dry powder particle formulations prepared by spray drying for delivery to the pulmonary system via inhalation. The invention comprises formulations and parameters for spray drying an RNA-containing powder, such as an mRNA-containing powder, with suitable chemical, physical and aerosol properties for delivery preferably using a dry powder inhaler system and preferably a patient-activated dry powder inhaler system, for example, the ARCUS® platform described at www.civitis.com. In the Examples, yeast RNA was used as a placeholder for mRNA.

Preferably, the respirable, RNA dry powder particle formulation for pulmonary delivery comprises:
i) at least about 1% RNA by weight of the particle;
ii) at least about 10% DPPC by weight of the particle;
iii) optionally, at least about 1% each of one or more of arginine, leucine, isoleucine, or valine by weight of the particle, wherein when two or more amino acids are selected, each amino acid has either the same or different weight percentage as the other selected amino acid(s);
iv) optionally, at least about 1% NaCl by weight of the particle;
v) optionally, at least about 10% Tris by weight of the particle;
vi) optionally, at least about 2% EDTA by weight of the particle;
vii) optionally, at least about 40% trehalose by weight of the particle;
viii) optionally, at least about 2% PEI by weight of the particle; and
ix) optionally, at least about 40% lactose by weight of the particle.
wherein all components of the RNA dry powder amount to 100 weight percent.

Preferably the formulation comprises about 1% to about 80%, and preferably about 1% to 70%, such as about 1%, 10%, 25%, 50%, or about 60% by weight of RNA.

Preferably the RNA can be at least about 10, 15, 16, 17, 18, 19, 20, 25, 30, 35, 40, 45, 50, 55, 60, 65, 70, 75, 80, 85, 90, 100, 125, 150, 200 or more nucleic acids in length. Preferably, the RNA may be from about 10 to about 12,000 nucleic acids or more in length. Preferably, the RNA may be from about 20 to about 12,000 or more nucleic acids in length. Preferably, the RNA may be from about 50 to about 12,000 or more nucleic acids in length. Preferably the RNA may be from about 100 to about 12,000 or more nucleic acids in length. Preferably the RNA can be from about 20 to about 12,000 monomers in length, or about 20 to about 10,000 monomers, or about 20 to 8,000 monomers, or about 20 to 6000 monomers, or about 20 to about 5000 monomers, or about 20 to about 4000 monomers, or about 20 to about 3600 monomers, or about 20 to about 3200 monomers, or about 20 to about 3000 monomers, or about 20 to about 2800 monomers, or about 20 to about 2600 monomers, or about 20 to 2400 monomers, or about 20 to 2200 monomers, or about 50 to 3200 monomers, or about 50 to 3000 monomers, or about 50 to 2600 monomers.

Preferably the formulation comprises about 10% to about 30%, and preferably about 15% to 25%, such as about 15%, 18%, 20%, 22%, or about 25% by weight of DPPC.

Preferably the formulation optionally comprises about 1% to about 90%, and preferably about 5% to 85%, such as about 5%, 20%, 30%, 40%, 50%, 55%, 70%, 74%, 79%, 80%, or about 85% by weight of one or more of arginine, leucine, isoleucine, or valine, wherein when two or more amino acids are selected, each amino acid has either the same or different weight percentage as the other selected amino acid(s).

Preferably the formulation optionally comprises about 1% to about 5%, and preferably about 1% to 3%, such as about 1%, 2%, or about 3% by weight of NaCl.

Preferably the formulation optionally comprises about 1% to about 25%, and preferably about 5% to 20%, such as about 5%, 10%, 15%, or about 20% by weight of Tris.

Preferably the formulation optionally comprises about 1% to about 10%, and preferably about 2% to 8%, such as about 2%, 3%, 4%, 5%, 6%, or about 7% by weight of EDTA.

Preferably the formulation further optionally comprises about 40% to about 90%, and preferably about 45% to 85%, such as about 45%, 50%, 52%, 53%, 54%, 55%, 60%, 65%, 70%, 72%, 74%, 75%, 77%, 79%, 80%, or about 82% by weight of trehalose.

Preferably the formulation further optionally comprises about 40% to about 90%, and preferably about 45% to 85%, such as about 45%, 50%, 52%, 53%, 54%, 55%, 60%, 65%, 70%, 72%, 74%, 75%, 77%, 79%, 80%, or about 82% by weight of lactose.

Preferably the formulation optionally comprises about 1% to about 10%, and preferably about 1% to 8%, such as about 1%, 2%, 3%, 4%, 5%, 6%, or about 7% by weight of PEI.

Preferably, the formulation is selected from the formulations listed in Table 1.

Weight percent is intended to reflect the total amount of solids, lipids, and/or excipients in the dry particles without regard to residual water, solvent or impurities. Preferably, all of the components of the dry particles amount to 100 wt %.

The respirable dry particles of the invention preferably have an MMAD of about 10 microns or less, such as an MMAD of about 0.5 micron to about 10 microns. Preferably, the dry particles of the invention have an MMAD of about 7 microns or less (e.g., about 0.5 micron to about 7 microns), preferably about 1 micron to about 7 microns, or about 2 microns to about 7 microns, or about 3 microns to about 7 microns, or about 4 microns to about 7 microns, or about 5 microns to about 7 microns, or about 1 micron to about 6 microns, or about 1 micron to about 5 microns, or about 2 microns to about 5 microns, or about 2 microns to about 4 microns, or about 3 microns.

The respirable dry particles of the invention preferably have a gPSD₅₀ of about 10 microns or less, such as a gPSD₅₀ of about 1 micron to about 7 microns. Preferably, the dry particles of the invention have a gPSD₅₀ of about 6 microns or less, such as about 1 micron to about 6 microns, preferably about 1 micron to about 5 microns, or about 1 micron to about 4 microns, or about 1 micron to about 3 microns, or about 1 micron to about 2 microns, or about 2 microns to about 5 microns, or about 2 microns to about 4 microns, or about 3 microns.

The fine particle fraction less than 5.6 microns of a powder, or FPF<5.6 μm of a powder, corresponds to the percentage of particles in the powder that have an aerodynamic diameter of less than 5.6 μm. The FPF<5.6 μm of a powder of the invention is preferably about 40% or more.

In certain embodiments, the FPF<5.6 μm of the powder is at least about 45%, 50%, 55%, 60%, 65%, 70%, 75%, or more. In one embodiment, the FPF<5.6 μm is about 40% to about 80%. In one embodiment, the FPF<5.6 μm is 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80%.

The fine particle fraction less than 3.4 microns of a powder, or FPF<3.4 μm of a powder, corresponds to the percentage of particles in the powder that have an aerodynamic diameter of less than 3.4 μm. In one embodiment, the FPF<3.4 μm of a powder of the invention is about 30% or more. In one embodiment, the FPF<3.4 μm of the powder is at least about 30%, 35%, 40%, 45%, 50%, 55%, 60%, 65%, 70%, or more. In one embodiment, the FPF<3.4 μm is about 40% to about 80%. In one embodiment, the FPF<3.4 μm is 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, or 80%.

Preferably, the powders of the invention have a tap density of less than about 0.4 g/cm³. For example, the powders have a tap density between 0.02 and 0.20 g/cm³, between 0.02 and 0.15 g/cm³, between 0.03 and 0.12 g/cm³, between 0.05 and 0.15 g/cm³, or less than about 0.15 g/cm³, or a tap density less than about 0.10 g/cm³, a tap density less than about 0.15 g/cm³. In one embodiment, the powders of the invention have a tap density of less than about 0.2 g/cm³. Preferably, the tap density is from about 0.02 to 0.175 g/cm³. Preferably, the tap density is from about 0.06 to 0.175 g/cm³.

Tap density can be measured by using instruments known to those skilled in the art such as the Dual Platform Microprocessor Controlled Tap Density Tester (Vankel, N.C.) or a GEOPYC™ instrument (Micrometrics Instrument Corp., Norcross, GA, 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which can contribute to low tap density include irregular surface texture and porous structure. The envelope mass density of an isotropic particle is defined as the mass of the particle divided by the minimum sphere envelope volume within which it can be enclosed. In one embodiment of the invention, the particles have an envelope mass density of less than about 0.4 g/cm³.

Preferably, the respirable dry powders and dry particles formulations of the invention have a water or solvent content of less than about 15% by weight, less than about 13% by weight, less than about 11.5% by weight, less than about 10% by weight, less than about 9% by weight, less than about 8% by weight, less than about 7% by weight, less than about 6% by weight, less than about 5% by weight, less than about 4% by weight, less than about 3% by weight, less than about 2% by weight, less than about 1% by weight or be anhydrous.

Preferably, the dry particle formulations of the invention can have a water or solvent content of less than about 6% and greater than about 1%, less than about 5.5% and greater than about 1.5%, less than about 5% and greater than about 2%, about 2%, about 2.5%, about 3%, about 3.5%, about 4%, about 4.5%, or about 5%.

### Treatment Indications.

The respirable polynucleotide formulations (e.g., mRNA formulations) described herein can be used in therapy. For example, a respirable polynucleotide formulation described herein can be administered via pulmonary administration to an animal or human patient, wherein polynucleotide is translated in vivo to produce a therapeutic peptide in the animal or subject. Accordingly, the respirable mRNA formulations of the invention may be used for treatment or prevention of disease or conditions in humans and other mammals.

The respirable mRNA formulations of the invention may be used for inducing translation of a synthetic or recombinant polynucleotide to produce a polypeptide in a cell population (e.g. epithelial cells of the lung). An effective amount of the formulation of the invention is based, at least in part, on the target tissue, target cell type, means of administration, physical characteristics of the nucleic acid (e.g., size, and extent of alternative nucleosides), and other determinants.

Therefore, the invention provides respirable formulations comprising polynucleotides (e.g., mRNA) for use in methods of inducing in vivo translation of a recombinant polypeptide in a mammalian subject in need thereof. Therein, an effective amount of an RNA in a respirable formulation has at least one translatable region encoding the desired polypeptide is administered to the subject using the delivery methods described herein. The mRNA is provided in the formulation in an amount and under other conditions such that the nucleic acid is localized into a cell or cells of the subject and the recombinant polypeptide is translated in the cell from the nucleic acid. The cell in which the nucleic acid is localized, or the tissue in which the cell is present, may be targeted with one or more than one rounds of nucleic acid administration.

The subject to whom the therapeutic agent is administered suffers from or is at risk of developing a disease, disorder, or deleterious condition. Provided are methods of identifying, diagnosing, and classifying subjects on these bases, which may include clinical diagnosis, biomarker levels, genome-wide association studies (GWAS), and other methods known in the art.

Preferably, the administered respirable polynucleotide formulations (e.g. mRNA formulations) results in the production of one or more recombinant polypeptides that provide a functional activity which is substantially absent in the cell in which the recombinant polypeptide is translated. For example, the missing functional activity may be enzymatic, structural, or gene regulatory in nature.

Preferably, the administered alternative nucleic acid directs production of one or more recombinant polypeptides that replace a polypeptide (or multiple polypeptides) that is substantially absent in the cell in which the recombinant polypeptide is translated. Such absence may be due to genetic mutation of the encoding gene or regulatory pathway thereof.

Preferably, the administered formulations direct production of antibodies, e.g., neutralizing antibodies, useful for modulating either directly or indirectly, the activity of a biological moiety present on the surface of or secreted from the cell.

The formulations of the invention also provide improved methods and compositions for the treatment of diseases related to protein deficiency using, for example, mRNA therapy to express a modified protein. The invention provides methods of treating a protein deficiency, comprising administering to a subject in need of treatment a formulation of the invention comprising, for example, an mRNA sequence described herein encoding a stabilized modified human protein or active fragments of such stabilized modified human protein at an effective dose and an administration interval such that at least one symptom or feature of the protein deficiency is reduced in intensity, severity, or frequency or has delayed onset. The formulations of the invention also provide modified proteins encoded by mRNA sequences wherein the modified proteins have improved properties such as enhanced stability and resistance to protein degradation and increased half-life as compared to wild type human proteins.

Preferably, the pulmonary administration of an mRNA composition described herein results in an increased therapeutic protein expression or activity of the subject as compared to a control level. Preferably, the control level is a baseline serum therapeutic protein expression or activity level in the subject prior to the treatment and/or the control level is indicative of the average serum protein expression or activity level in patients without treatment.

Preferably, the proteins encoded by the mRNA formulations described herein are produced from a heterologous mRNA construct comprising an open reading frame (ORF) also referred to herein as a “coding sequence” (CDS) encoding for a therapeutic protein. Preferably, the coding sequence is codon optimized. Preferably, coding sequence is optimized to have a theoretical minimum of uridines possible to encode for a therapeutic protein. Preferably, the mRNA constructs described herein comprise one or more of the following features: a 5′ cap; a 5′UTR, a 5′UTR enhancer sequence, a Kozak sequence or a partial Kozak sequence, a 3′UTR, an open reading frame encoding a therapeutic protein and a poly A tail. Preferably, the mRNA constructs described herein can provide high-efficiency expression of a modified protein. The expression can be in vitro, ex vivo, or in vivo.

Preferably, an mRNA described herein comprises a Kozak sequence and/or a 3′UTR. As is understood in the art, a Kozak sequence is a short consensus sequence centered around the translational initiation site of eukaryotic mRNAs that allows for efficient initiation of translation of the mRNA. The ribosomal translation machinery recognizes the AUG initiation codon in the context of the Kozak sequence. A Kozak sequence, may be inserted upstream of the coding sequence for the therapeutic protein of interest, downstream of a 5′ UTR or inserted upstream of the coding sequence for the therapeutic protein of interest and downstream of a 5′ UTR. Preferably, an mRNA described herein comprises a 3′ tail region, which can serve to protect the mRNA from exonuclease degradation. The tail region may be a 3′poly(A) and/or 3′poly(C) region. Preferably, the tail region is a 3′ poly(A) tail. As used herein a “3′ poly(A) tail” is a polymer of sequential adenine nucleotides that can range in size from, for example 10 or more adenosines.

3′ Poly(A) tails can be added using a variety of methods known in the art, e.g., using poly(A) polymerase to add tails to synthetic or in vitro transcribed RNA. Other methods include the use of a transcription vector to encode poly A tails or the use of a ligase (e.g., via splint ligation using a T4 RNA ligase and/or T4 DNA ligase), wherein poly(A) may be ligated to the 3′ end of a sense RNA. Preferably, a combination of any of the above methods is utilized.

Preferably, an mRNA formulation described herein comprises a 5′ cap. 5′-ends capped with various groups and their analogues are known in the art. The 5′ cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003). The 5′ cap may be an ARCA cap (3′-OMe-m7G(5′)pppG). The 5′ cap may be an mCAP (m7G(5′)ppp(5′)G, N⁷-Methyl-Guanosine-5′-Triphosphate-5′-Guanosine). The 5′ cap may be resistant to hydrolysis.

Preferably an mRNA formulation described herein comprises one or more chemically modified nucleotides. Examples of nucleic acid monomers include non-natural, modified, and chemically modified nucleotides, including any such nucleotides known in the art. mRNA sequences comprising chemically modified nucleotides have been shown to improve mRNA expression, expression rates, half-life and/or expressed protein concentrations. mRNA sequences comprising chemically modified nucleotides have also been useful to optimize protein localization thereby avoiding deleterious bio-responses such as the immune response and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N⁴,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N⁴,N⁴-dimethylcytidine.

Examples of modified or chemically modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N⁶-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N⁶-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N⁶,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically modified nucleotides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically modified nucleotides include N¹-methylguanosine, N²-methylguanosine, thienoguanosine, 7-deazaguanosine, 8-oxoguanosine, 8-bromoguanosine, O⁶-methylguanosine, xanthosine, inosine, and N¹-methylinosine.

Examples of modified or chemically modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically modified nucleotide monomers include 2′,4′-constrained 2′-O-methoxyethyl (cMOE) and 2′-O-Ethyl (cEt) modified DNAs.

Examples of modified and chemically modified nucleotide monomers include 2′-amino nucleotides, 2′-O-amino nucleotides, 2′-C-allyl nucleotides, and 2′-O-allyl nucleotides.

Examples of modified and chemically modified nucleotide monomers include N⁶-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically modified nucleotide monomers include replacing the 2′-OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically modified nucleotide monomers may be found in nature.

Preferred nucleotide modifications include N¹-methylpseudouridine and 5-methoxyuridine.

The formulations of the invention may also comprise polynucleotides (e.g. DNA, RNA, cDNA, mRNA) encoding a modified human protein of interest that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

Preferably, a polynucleotide described herein encoding a modified therapeutic protein described herein can invoke an immune response or be used in a gene therapy. Gene therapy is a technique that uses genes to treat or prevent disease and can be used to treat a disorder by inserting a gene into a subject's cells. In some embodiments a polynucleotide encoding a modified therapeutic protein described herein replaces a mutated gene that causes disease. In other embodiments, a polynucleotide encoding a modified therapeutic protein described herein is used to inactivate, or “knock out,” a mutated gene that is functioning improperly. In yet other embodiments, a polynucleotide encoding a modified therapeutic protein described herein introduces a new gene into a subject to help fight a disease.

An effective dose of an mRNA formulation of the can be an amount that is sufficient to treat protein deficiency in a cell and/or in a patient. A therapeutically effective dose can be an amount of an agent or formulation that is sufficient to cause a therapeutic effect. A therapeutically effective dose can be administered in one or more separate administrations, and by different routes. Generally, a therapeutically effective amount is sufficient to achieve a meaningful benefit to the subject (e.g., treating, modulating, curing, preventing and/or ameliorating phenylketonuria). For example, a therapeutically effective amount may be an amount sufficient to achieve a desired therapeutic and/or prophylactic effect. Generally, the amount of a therapeutic agent administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of a respirable mRNA formulation described herein. Respirable formulations comprising an mRNA sequence encoding an ORF protein described herein can be administered at regular intervals, depending on the nature, severity and extent of the subject's condition. Preferably, a therapeutically effective amount a respirable mRNA formulation of the may be administered periodically at regular intervals (e.g., once every year, once every six months, once every four months, once every three months, once every two months, once a month), biweekly, weekly, daily, twice a day, three times a day, four times a day, five times a day, six times a day, or continuously.

The respirable polynucleotide formulations of the invention may also be used for intracellular expression of, for example, an antibody, wherein the RNA (sequence) codes for an antibody or contains at least one coding region, which codes for at least one antibody, respectively. An antibody-coding RNA according to the invention includes any RNA which encodes an antibody. More generally, the RNA of the present invention (directed to intracellular expression) contains at least one coding region, wherein the at least one coding region codes for at least one antibody. If more than one coding region is contained in the RNA molecule of the invention, the second, third etc. coding region may code for antibodies as well, which may be the same or distinct from the first antibody coding region. Preferably, the RNA comprising the formulations of the invention contains at least two coding regions, all of them coding for identical or distinct antibodies. Preferably, the RNA of a formulation may code for more than one antibody within the same coding region.

The antibody-coding RNA of the formulation of the invention can be single-stranded or double-stranded, linear or circular, or most preferably, in the form of mRNA. The antibody-coding RNA is preferably in the form of single-stranded RNA, even more preferably in the form of mRNA.

An antibody-coding RNA according to the invention preferably has a length of from 50 to 15,000 nucleotides, more preferably a length of from 50 to 10,000 nucleotides, even more preferably a length of from 500 to 10,000 nucleotides and most preferably a length of from 500 to 7,000, 500 to 5,000 or 700 to 3,000 nucleotides.

The antibodies coded by the respirable RNA formulations according to the invention can be chosen from all antibodies, e.g. from all antibodies which are generated by recombinant methods or are naturally occurring and are known to a person skilled in the art from the prior art, in particular antibodies which are (can be) employed for therapeutic purposes or for diagnostic or for research purposes or have been found with particular diseases, e.g., cancer diseases, infectious diseases.

Antibodies which are coded by an RNA formulation of the invention typically include all antibodies (described above) which are known to a person skilled in the art, e.g. naturally occurring antibodies or antibodies generated in a host organism by immunization, antibodies prepared by recombinant methods which have been isolated and identified from naturally occurring antibodies or antibodies generated in a host organism by (conventional) immunization or have been generated with the aid of molecular biology methods, as well as chimeric antibodies, human antibodies, humanized antibodies, bispecific antibodies, intrabodies, i.e., antibodies expressed in cells and possibly localized in particular cell compartments, and fragments of the abovementioned antibodies. Insofar, the term antibody is to be understood in its broadest meaning. In this context, antibodies in general typically comprise a light chain and a heavy chain, both of which have variable and constant domains. The light chain comprises the N-terminal variable domain VL and the C-terminal constant domain CL. The heavy chain of an IgG antibody, in contrast, can be divided into an N-terminal variable domain VH and three constant domains C_{H}1, C_{H}2 and C_{H}3.

Antibodies which are coded by RNAs according to the invention particularly preferably include so-called full-length antibodies, i.e. antibodies which comprise both the complete heavy and the complete light chains, as described above. RNAs which alternatively code for one or more antibody fragment(s) of the antibodies described above, instead of the corresponding full-length antibody, can furthermore be provided in the context of the present invention. Examples of such antibody fragments are any antibody fragments known to a person skilled in the art, e.g. Fab, Fab′, F(ab′)₂, Fc, Facb, pFc′, Fd, and Fv fragments of the abovementioned antibodies etc.

Without being limited thereto, respirable formulations comprising RNAs which code for antibodies inter alia code for those antibodies which bind antigens or specific nucleic acids. Antigens are typically molecules which are recognized as exogenous by the immune system and conventionally cause an immune reaction or immune response with the formation of antibodies directed specifically against them. However, antigens can also include, especially in the case of autoimmune diseases, endogenous molecules or structures which are incorrectly recognized as exogenous by the immune system and thereby trigger an immune reaction.

Antigens typically comprise proteins, peptides or epitopes of these proteins or peptides. In this context, epitopes (also called “antigenic determinants”) are typically small regions (molecular sections) lying on the surface of such protein or peptide structures and having a length of from 5 to 15, in rare case also to 25, preferably 6 to 9 amino acids. Antigens can furthermore also include lipids, carbohydrates etc. In the context of the present invention, antigens also include, for example, so-called immunogens, i.e. antigens which lead to an immunity of the organism transfected therewith. Antigens by way of example include, without being limited thereto, surface antigens of cells, tumor antigens etc. For example, according to the present invention antibodies can bind the following antigens (which typically occur in vertebrates), e.g. tumor-specific surface antigens (TSSA), e.g. 5T4, α5β1-integrin, 707-AP, AFP, ART-4, B7H4, BAGE, β-catenin/m, Bcr-abl, MN/C 1X-antigen, CA125, CAMEL, CAP-1, CASP-8, CD4, CD19, CD20, CD22, CD25, CDC27/m, CD 30, CD33, CD52, CD56, CD80, CDK4/m, CEA, CT, Cyp-B, DAM, EGFR, ErbB3, ELF2M, EMMPRIN, EpCam, ETV6-AML1, G250, GAGE, GnT-V, Gp100, HAGE, HER-2/neu, HLA-A*0201-R170I, HPV-E7, HSP70-2M, HAST-2, hTERT (or hTRT), iCE, IGF-1R, IL-2R, IL-5, KIAA0205, LAGE, LDLR/FUT, MAGE, MART-1/Melan-A, MART-2/Ski, MCiR, myosin/m, MUC1, MUM-1, -2, -3, NA88-A, NY-ESO1, PAP, proteinase-3, p190 minor bcr-abl, Pml/RARα, PRAME, PSA, PSM, PSMA, RAGE, RU1 or RU2, SAGE, SART-1 or SART-3, survivin, TEL/AML1, TGFβ, TPI/m, TRP-1, TRP-2, TRP-2/1NT2, VEGF and WT1, or sequences, such as e.g. NY-Eso-1 or NY-Eso-B. Other antigen targets immune check point inhibitor proteins including CTLA4 and PD-1 or PD1 ligands.

Tumor antigens can, for example, typically be responsible for metastasis. In this context, such tumor antigens which cause modified cell-cell interactions compared with the native state are of interest in particular.

As discussed previously with regard to protein replacement therapy, an antibody coding RNA respirable formulation of the invention may comprise modified RNA for any purpose such as increasing expression, improved stabilization of secondary structure where necessary, stabilize the RNA against degradation, and reducing immunogenicity of the respirable RNA.

Preferably, diseases of the respiratory system which are treatable using the formulations of the invention include, but are not limited to, asthma, pulmonary arterial hypertension, chronic obstructive pulmonary disease (COPD), respiratory distress syndrome (RDS), chronic bronchitis, acute bronchitis, emphysema, cystic fibrosis, pneumonia, tuberculosis, lung cancer, acute respiratory distress syndrome (ARDS), influenza, respiratory syncytial virus, pneumoconiosis, interstitial lung disease (ILD) (such as sarcoidosis, idiopathic pulmonary fibrosis, and autoimmune disease), pulmonary embolism, pleural effusion, and mesothelioma.

### EXAMPLES

The present invention will be better understood in connection with the following Examples. However, it should be understood that these examples are for illustrative purposes only and are not meant to limit the scope of the invention. Various changes and modifications will be apparent to those skilled in the art and such changes and modifications including, without limitation, those relating to the formulations and/or methods of the invention may be made without departing from the spirit of the invention and the scope of the appended claims.

### Initial Development

Studies R1 (Batch #449043), R2 (Batch #483016), and R3 (Batch #483022) were done as part of the initial development work for producing RNA delivery powder. These three studies aimed to see if it was possible to spray dry a powder containing a load of yeast RNA (as a placeholder for mRNA) with suitable physical and aerosol properties for delivery. By varying the formulation and parameters used to spray dry the RNA powder, it was found that suitable powder could be produced when using a product filter bag pre-conditioned in a dry 15% RH environment.

In Study R2, the excipients were varied from R1 in order to assess if the combination of excipients had caused the powder particle in R1 to stick to the product filter bag. A sugar, Trehalose, took the place of the amino acid previously used. Additionally, EDTA and Tris buffer replaced NaCl because they were thought to help with faster dissolution of RNA into the aqueous phase. The RNA load was kept constant at 10%. During the spray drying of these components, powder was partially released upon pulsing the product filter bag. After approximately 100 minutes of spray drying enough powder stuck to the bag to cause the system pressure to become positive. Therefore, the run was ended early. The collected powder was found to have suitable aerosol and physical properties for use in a dry powder inhaler system and particularly for use in the ARCUS® platform, with an FPF<5.6 μm of 61% and a gPSD₅₀ (average geometric particle size in microns (d50)) of 3.3 μm.

In Study R3, a similar formulation to that of R2 was run. The major change from the previous study was that the product filter bag was condition in a dry 15% RH environment immediately prior to the run. The conditioning of the bag resulted in better release of the powder particles from the bag, which results in the powder being successfully collected as product. This boosted the yield significantly. The resulting powder had similar properties to that of R2, with an FPF<5.6 μm of 61% and a gPSD₅₀ of 2.0 μm.

Spraying RNA with Trehalose and Leucine

Studies R4 (Batch #483028) and R5 (Batch #483045) were formulated using either the amino acid Leucine or the sugar Trehalose, respectively, as the main excipients. Both of these excipients have been used extensively in past spray drying runs, and tend to produce powders with suitable physical and aerosol properties. R4 and R5 studies were done to give initial confirmation that RNA could also be spray dried with these excipients to produce powder with desirable properties.

Studies R4 and R5 yielded powders with desirable properties, thus confirming that RNA spray dried well with these excipients. R4 produced a powder with an FPF<5.6 μm of 75% and a gPSD₅₀ of 2.5 μm. R5 produced a powder with an FPF<5.6 μm of 71% and a gPSD₅₀ of 3.8 μm. Additional powder properties are presented in Examples 4 and 5 below.

### Increasing RNA Load

Studies R5 (Batch #483045) and R9 (Batch #483085) were formulated using Trehalose as the main excipient and had 1% and 25% RNA, respectively. Studies R11 (Batch #483109) and R14 (Batch #483132) were formulated using Isoleucine as the main excipient and had 50% and 25% RNA, respectively. In both cases, increasing the RNA load in the particles increased the residual solvent content. In studies R5 and R9 the TGA−120° C. (TGA-120: total weight loss or volatiles from heating a sample up to 120° C. at a heating ramp of 20° C./min) increased from 2.93% to 3.50%, and in R14 and R11 the residual solvent content increased from 1.73% to 3.55%. In formulations with higher loads of RNA the weight continued to decrease after 120° C. on the TGA. This was also observed when pure yeast RNA was run on the TGA.

The FPF<5.6 um and <3.4 um decreased with increasing RNA load. This change was more dramatic when Isoleucine was the main excipient, decreasing from 60% to 49% for FPF <5.6 um and decreasing from 48% to 38% for FPF<3.4 um. The FPF for Trehalose did not considerably decrease when the RNA load was increased from 1% to 25%.

### Addition of Charged Compound to Formulations

Several studies, including R1 (Batch #449043), R6 (Batch #483071), and R12 (Batch #483119) have been made with positively charged excipients.

Reducing RNA's Maximum Solubility with PEI

In preparation for running Study R6 (Batch #483071) 1% RNA, 74% Trehalose, and 2% NaCl were dissolved in water. After the 3 compounds were dissolved, 5% PEI was added to the aqueous phase. The PEI successfully dissolved and Study R6 proceeded. When 5% PEI was added to the aqueous phase of Study R8 (Batch #483084), which was composed of 10% RNA, 74% Trehalose, and 2% NaCl, the RNA precipitated. This indicates that the presence of PEI reduces RNA's maximum solubility. This could be used in later studies to create larger, more porous particles by causing the RNA to precipitate out of solution after atomization.

### Varying Product Collection Method

The formulations and process parameters for Studies R6 (Batch #483071), R7 (Batch #483072), and R10 (Batch #483107) are nearly identical except for how the product was collected. (See Examples 1 and 3 below) R6 used the filter baghouse set up described below in Example 2 (Equipment Setup) and Example 3 (Process Parameters). R7 and R10 used cyclones to collect the final product. This was done in order to determine if the time required to begin collecting powder could be decreased.

The time required to prime the system decreased for batches with the cyclones because the filter baghouse and product filter no longer had to be coated before the powder would start to accumulate in the collection vessel. It only took 7 and 12 minutes for powder to start collecting in R7 and R10, respectively. For batch R6 the system had to be run for about 30 minutes before power began to be collected.

In R7 and R10 the bulk of the smaller particles were not deposited in the collection vessel. The smaller particles instead exited through the top of the cyclone and flowed to the filter baghouse. This caused the yield to decrease from 23.8% in R6 to 12.8% and 15.2% in R7 and R10, respectively. (See Table 6 in Example 5 below) This also explains the increase in the gPSD₅₀ and the decrease in the FPF in R7 and R10 in comparison to R6.

There were no major changes in the solid-state characteristics of the powders that were collected with the cyclone, as opposed to the filter baghouse.

### Example 1-Materials and Methods

Formulations used in the studies investigating formulation of dry powder for delivery of RNA can be found listed in Table 19.

TABLE 19

| No. | Batch # | Formulation |
|---|---|---|
| R1 | 449043 | RNA:Arginine:DPPC:NaCl (10:70:18:2) |
| R2 | 483016 | RNA:Trehalose:DPPC:Tris:EDTA (10:53:18:15:4) |
| R3 | 483022 | RNA:Trebalose:DPPC:Tris:EDTA (10:52:18:15:5) |
| R4 | 483028 | RNA:Leucine:DPPC:NaCl (1:79:18:2) |
| R5 | 483045 | RNA:Trehalose:DPPC:NaCl (1:79:18:2) |
| R6 | 483071 | RNA:Trehalose:DPPC:PEI:NaCl (1:74:18:5:2) |
| R7 | 483072 | RNA:Trehalose:DPPC:PEI:NaCl (1:74:18:5:2) |
| R8 | 483084 | RNA:Trehalose:DPPC:PEI:NaCl (10:65:18:5:2) |
| R9 | 483085 | RNA:Trehalose:DPPC:NaCl (25:55:18:2) |
| R10 | 483107 | RNA:Trehalose:DPPC:PEI:NaCl (1:74:18:5:2) |
| R11 | 483109 | RNA:Isoleucine:DPPC:NaCl (50:30:18:2) |
| R12 | 483119 | RNA:Valine:DPPC:Arginine:NaCl (25:50:18:5:2) |
| R13 | 483120 | RNA:Valine:DPPC:Arginine:NaCl (25:50:18:5:2) |
| R14 | 483132 | RNA:Isoleucine:DPPC:NaCl (25:55:18:2) |

Materials used in the studies investigating formulation of dry powder for delivery of RNA can be found listed in Table 2.

TABLE 2

| Chemical | Abbreviation |
|---|---|
| Yeast ribonucleic acid | RNA |
| Dipalmitoylphosphatidylcholine | DPPC |
| Sodium chloride | NaCl |
| Trehalose | — |
| Arginine | Arg |
| Isoleucine | Ile |
| Leucine | Leu |
| Valine | Val |
| Tris(hydroxymethyl)aminomethane | Tris |
| Ethylenediaminetetraacetic acid | EDTA |
| Polyethylenimine | PEI |

Manufacturers of the reagents used in the studies are listed immediately below.

Reagents
1. RNA (Roche Diagnostics GmbH, Mannheim, Germany)
2. DPPC (Lipoid GmbH, Steinhausen, Switzerland)
3. NaCl (BDH, VWR, Radnor, PA, USA)
4. Trehalose (Sigma Aldrich, St. Louis, MO, USA)
5. Arginine (Amresco Life Sciences, VWR, Radnor, PA, USA)
6. Isoleucine (Sigma Aldrich, St. Louis, MO, USA)
7. Leucine (Sigma Aldrich, St. Louis, MO, USA)
8. Valine (Sigma Aldrich, St. Louis, MO, USA)
9. Tris (hydroxymethyl)aminomethane (G Biosciences, St. Louis, MO, USA)
10. Ethylenediaminetetraacetic acid (BDH, VWR, Radnor, PA, USA)
11. Polyethylenimine (Aldrich Chemistry, Sigma, St. Louis, MO, USA).

### Example 2-Equipment Setup

The spray dryer used for this evaluation comprised a size 1 spray dryer (GEA, NIRO PSD1, Dusseldorf, Germany) equipped with a single-bag product filter bag-house. The product filter bag with this single-bag product filter baghouse was model number P034582-016-210 (Donaldson Filtration Solutions, Bloomington, MN) and is made of polyester with PTFE bag (5.87″ diameter×37.50″ length) and has a flat width of 9.23″. The air cap used for this evaluation was model number 67147 (Spraying Systems Co., Wheaton, IL) and the fluid cap used for this evaluation was model number 2850 (Spraying Systems Co., Wheaton, IL).

### Example 3-Spray Drying Process Parameters

The powders were made using a size 1 spray dryer (GEA, NIRO PSD1, Dusseldorf, Germany). Table 3 lists the typical process parameter values used for the production of powders containing RNA. Process parameters for each specific run are listed in Table 4.

TABLE 3

| Parameter | Target Value |
|---|---|
| Inlet temperature (° C.) | 67 |
| Outlet temperature (° C.) | 40 |
| Drying gas flow rate (kg/hr) | 125 |
| Atomization gas flow rate (kg/hr) | 30 |
| Aqueous flow rate (mL/min) | 15 |
| Organic flow rate (mL/min) | 15 |
| System pressure (“WC) | −2.0 |
| Filter bag pulse time (sec) | 30 |
| Pulse tank pressure (psi) | 70 |
| Baghouse purge rate (scfh) | 20 |
| Total solid concentration (g/L) | 4 |

TABLE 4

| Parameter | R1 | R2 | R3 | R4 | R5 | R6 | R7 | R8 | R9 | R10 | R11 | R12 | R13 | R14 |
|---|---|---|---|---|---|---|---|---|---|---|---|---|---|---|
| Inlet temperature (° C.) | 67 | 67 | 67 | 67 | 67 | 67 | 67 | N/A | 67 | 67 | 67 | 67 | 67 | 67 |
| Outlet temperature (° C.) | 40 | 40 | 40 | 40 | 40 | 40 | 40 | N/A | 40 | 40 | 40 | 40 | 40 | 40 |
| Drying gas flow rate | 125 | 125 | 125 | 125 | 125 | 125 | 125 | N/A | 125 | 125 | 125 | 125 | 125 | 125 |
| (kg/hr) | | | | | | | | | | | | | | |
| Atomization gas flow rate | 40 | 40 | 35 | 30 | 25 | 30 | 30 | N/A | 30 | 30 | 30 | 30 | 30 | 30 |
| (kg/hr) | | | | | | | | | | | | | | |
| Aqueous flow rate | 15 | 15 | 15 | 15 | 15 | 15 | 15 | N/A | 15 | 15 | 15 | 15 | 15 | 15 |
| (mL/min) | | | | | | | | | | | | | | |
| Organic flow rate | 15 | 15 | 15 | 15 | 15 | 15 | 15 | N/A | 15 | 15 | 15 | 15 | 15 | 15 |
| (mL/min) | | | | | | | | | | | | | | |
| System pressure (″WC) | −2.0 | −2.0 | −2.0 | −2.0 | −2.0 | −2.0 | −2.0 | N/A | −2.0 | −2.0 | −2.0 | −2.0 | −2.0 | −2.0 |
| Filter bag pulse time (sec) | 30 | 30 | 30 | 30 | 30 | 30 | 1200 | N/A | 30 | 600 | 30 | 30 | 30 | 30 |
| | | | | | | | (twice) | | | | | | | |
| Pulse tank pressure (psi) | 70 | 70 | 80 | 70 | 70 | 70 | 70 | N/A | 70 | 70 | 70 | 70 | 70 | 70 |
| Baghouse purge rate | 20 | 20 | 20 | 20 | 20 | 20 | N/A | N/A | 20 | 20 | 20 | 20 | 20 | 20 |
| (scfh) | | | | | | | | | | | | | | |
| Total solid concentration | 4 | 4 | 4 | 4 | 4 | 4 | 4 | N/A | 4 | 4 | 4 | 4 | 4 | 4 |
| (g/L) | | | | | | | | | | | | | | |
| Product collection method | FBH | FBH | FBH | FBH | FBH | FBH | Cyclone | N/A | FBH | Small | FBH | FBH | FBH | FBH |
| | | | | | | | | | | Cyclone | | | | |

Legend:
FBH: Filter Baghouse; The cyclones were located between the pressure sensor at the exit of the spray dryer and the filter baghouse.

### Example 4—Stability Data for Spray Dried Powders with RNA

Stability data for the spray dried powders with RNA are presented in Table 5.

TABLE 5

| | | Conditions | | gPSD₅₀ | Size 00 FPF | Size 00 FPF | | TGA-120° C. | | |
|---|---|---|---|---|---|---|---|---|---|---|
| Study | Formulation | (40° C./75% RH) | | (um) | <5.6 um (%) | <3.4 um (%) | XRPD | (%) | Low T1 (° C.) | Low T2 (° C.) |
| R1 | RNA:Arginine:DPPC: | t = 0 | | No powder was collected at the end of the run | | | | | | |
| | NaCl | | | | | | | | | |
| | (10:70:18:2) | | | | | | | | | |
| R2 | RNA:Trehalose:DPPC:Tris: | t = 0 | | 3.3 | 61 | 46 | NT | NT | NT | NT |
| | EDTA (10:53:18:15:4) | t = 2 week | ND | NT | 24 | 16 | A-8 | 2.71 | 40.6 | 52.2, 63.3 |
| | | | SG | NT | 58 | 42 | A-8 | 1.68 | 38.7 | 55.3, 64.5 |
| | | t = 1 month | ND | NT | 29 | 22 | A-9 | 2.71 | 42.0 | 52.3 |
| | | | SG | NT | 65 | 49 | A-9 | 1.23 | 36.0 | 55.8, 65.5 |
| | | t = 3 month | ND | NT | 17 | 15 | A-10 | NT | 37.2 | 53.12 |
| | | | SG | NT | 61 | 45 | A-10 | 1.26 | NT | NT |
| R3 | RNA:Trehalose:DPPC:Tris: | t = 0 | | 2.0 | 61 | 50 | A-3 | 2.67 | 42.4 | 52.8, 62.4, |
| | EDTA (10:52:18:15:5) | | | | | | | | | 73.6, 80.2 |
| | | t = 2 week | ND | NT | 45 | 30 | A-3 | 2.48 | 42.9 | 54.1, 62.4, |
| | | | | | | | | | | 66.3 |
| | | | SG | NT | 60 | 49 | A-3 | 1.76 | 39.2 | 57.1, 65.6 |
| | | t = 1 month | ND | NT | 36 | 25 | A-3 | 2.63 | 42.1 | 53.0, 61.4 |
| | | | SG | NT | 62 | 49 | A-3 | 1.30 | 50.0 | 55.7, 65.8 |
| | | t = 3 month | ND | NT | 23 | 17 | A-3 | 2.77 | 41.6 | 54.1 |
| | | | SG | NT | 54 | 43 | A-3 | 1.64 | 37.2 | 56.0, 65.4 |
| | | t = 6 month | ND | NT | 4 | 3 | A-3 | 2.70 | 44.9 | 56.0, 66.0 |
| | | | SG | NT | 73 | 60 | A-3 | 1.68 | 41.2 | 54.1 |
| R4 | RNA:Leucine:DPPC: | t = 0 | | 2.5 | 75 | 56 | A-6 | 0.26 | none | 72.2, 78.3, |
| | NaCl | | | | | | | | | 84.5, 89.3 |
| | (1:79:18:2) | t = 2 week | ND | NT | 65 | 51 | A-6 | 0.25 | none | 72.4, 79.2 |
| | | | SG | NT | 73 | 59 | A-6 | 0.20 | 57.7 | 73.1, 79.5 |
| | | t = 1 month | ND | NT | 68 | 55 | A-6 | 0.31 | none | 71.9, 78.7, |
| | | | | | | | | | | 102.5 |
| | | | SG | NT | 68 | 54 | A-6 | 0.21 | none | 72.3, 78.7, |
| | | | | | | | | | | 93.7 |
| | | t = 3 month | ND | NT | 63 | 47 | A-6 | 0.225 | 59.7 | 70.6, 77.4 |
| | | | SG | NT | 66 | 52 | A-6 | 0.192 | none | 72.7, 79.7 |
| | | t = 6 month | ND | NT | 61* | 44* | A-6 | .0150 | none | 68.9, 75.1 |
| | | | SG | NT | 66 | 52 | A-6 | 0.195 | none | 72.2, 79.4 |
| R5 | RNA:Trehalose:DPPC: | t = 0 | | 3.8 | 71 | 55 | A-3 | 2.93 | 51.9 | 59.0, 69.2 |
| | NaCl | t = 2 week | ND | NT | 63 | 49 | A-3 | 3.00 | NT | NT |
| | (1:79:18:2) | | SG | NT | 69 | 54 | A-12 | 2.06 | NT | NT |
| | | t = 1 month | ND | NT | 64 | 50 | A-3 | 3.00 | none | 60.8, 72.5 |
| | | | SG | NT | 68 | 53 | A-3 | 2.16 | none | 66.2, 78.2 |
| | | t = 3 month | ND | NT | 56 | 42 | A-3 | 3.01 | none | 61.0, 73.7 |
| | | | SG | NT | 62 | 52 | A-3 | 2.20 | none | 65.2, 79.3 |
| | | t = 6 month | ND | NT | 65 | 48 | A-3 | 2.89 | none | 62.6, 75.1 |
| | | | SG | NT | 69 | 54 | A-3 | 2.09 | none | 66.6, 81.5 |
| R6 | RNA:Trehalose: DPPC: | t = 0 | | 3.3 | 77 | 63 | A-3 | 3.22 | 50.0 | 56.3, 65.3 |
| | PEI:NaCl | t = 2 week | ND | NT | 63 | 49 | A-3 | 3.13 | 46.7 | 57.0, 65.1 |
| | (1:74:18:5:2) | | SG | NT | 76 | 62 | A-3 | 2.26 | none | 63.7, 74.2 |
| | | t = 1 month | ND | NT | 63 | 48 | A-3 | 2.89 | 49.1 | 57.5, 65.4 |
| | | | SG | NT | 70 | 56 | A-3 | 2.19 | none | 64.3, 75.5 |
| | | t = 3 month | ND | NT | 58 | 42 | A-3 | 2.94 | 48.9 | 55.2, 62.4 |
| | | | SG | NT | 72 | 56 | A-3 | 1.96 | none | 62.78, 76.43 |
| | | t = 6 month | ND | NT | 52* | 38* | A-3 | 3.20 | none | 56.5, 63.0 |
| | | | SG | NT | 70 | 60 | A-3 | 2.08 | none | 64.9, 78.9 |
| R7 | RNA:Trehalose:DPPC: | t = 0 | | 5.0 | 56 | 36 | A-3 | 3.89 | 49.1 | 55.1, 66.2 |
| | PEI:NaCl | t = 2 week | ND | NT | 53 | 33 | A-3 | 3.27 | 47.6 | 57.4, 62.9, |
| | (1:74:18:5:2) | | | | | | | | | 66.6 |
| | | | SG | NT | 46 | 30 | A-3 | 2.20 | none | 63.6, 74.4 |
| | | t = 1 month | ND | NT | 50 | 28 | A-3 | 3.21 | 49.7 | 57.8, 62.9, |
| | | | | | | | | | | 67.2 |
| | | | SG | NT | 48 | 30 | A-3 | 2.10 | none | 63.2, 75.7 |
| | | t = 3 month | ND | NT | 52 | 31 | A-3 | 3.89 | 46.5 | 56.0, 60.9, |
| | | | | | | | | | | 66.0 |
| | | | SG | NT | 54 | 33 | A-3 | 2.23 | none | 64.7, 78.1 |
| | | t = 6 month | ND | NT | 43 | 27 | A-3 | 3.51 | 46.4 | 55.0, 66.2 |
| | | | SG | NT | 42 | 27 | A-3 | 2.35 | none | 63.0, 78.1 |
| R8 | RNA:Trehalose:DPPC: | t = 0 | | Run was not performed because the addition of PEI caused the RNA to crash out of solution | | | | | | |
| | PEI:NaCl (10:65:18:5:2) | | | | | | | | | |
| R9 | RNA:Trehalose:DPPC: | t = 0 | | 2.7 | 68 | 54 | A-3 | 3.50 | 50.7 | 68.3 |
| | NaCl | t = 2 week | ND | NT | 59 | 45 | A-3 | 3.18 | none | 69.9, 76.6 |
| | (25:55:18:2) | | SG | NT | 68 | 52 | A-3 | 2.85 | 55.4 | 71.0, 78.4 |
| | | t = 1 month | ND | NT | 63 | 51 | A-3 | 2.92 | none | 70.3, 78.4 |
| | | | SG | NT | 70 | 55 | A-3 | 2.55 | 48.2 | 56.4, 71.8, |
| | | | | | | | | | | 81.5 |
| | | t = 3 month | ND | NT | 66 | 51 | A-3 | 2.98 | none | 70.2, 80.2 |
| | | | SG | NT | 73 | 56 | A-3 | 2.56 | 55.4 | 71.5, 83.6 |
| R10 | RNA:Trehalose:DPPC: | t = 0 | | 4.9 | 54 | 38 | A-3 | 3.96 | 49.8 | 54.8, 66.4 |
| | PEI:NaCl | t = 2 week | ND | NT | 56 | 37 | A-3 | 5.54 | 45.0 | 56.9, 66.4 |
| | (1:74:18:5:2) | | SG | NT | 59 | 41 | A-3 | 1.96 | none | 64.2, 74.4 |
| | | t = 1 month | ND | NT | 57 | 38 | A-3 | 4.44 | 45.5 | 55.4, 65.3 |
| | | | SG | NT | 48 | 34 | A-3 | 2.37 | none | 63.4, 75.3 |
| | | t = 3 month | ND | NT | 60 | 37 | A-3 | 3.38 | 46.7 | 55.0, 66.0 |
| | | | SG | NT | 63 | 43 | A-3 | 2.21 | none | 63.7, 77.4 |
| | | t = 6 month | ND | NT | 51 | 32 | A-3 | 3.50 | none | 55.0, 65.6 |
| | | | SG | NT | 49 | 31 | A-3 | 2.38 | none | 63.5, 77.9 |
| R11 | RNA:Isoleucine:DPPC: | t = 0 | | 2.2 | 49 | 34 | A-3 | 3.55 | 25.6 | 50.4, 62.9 |
| | NaCl | t = 2 week | ND | NT | 39 | 24 | A-3 | 2.93 | 62.2 | 87.0 |
| | (50:30:18:2) | | SG | NT | 43 | 29 | A-11 | 2.55 | 53.0 | 65.4, 89.9 |
| | | t = 1 month | ND | NT | 36 | 19 | A-11 | 2.85 | 55.2 | 67.1, 70.3, |
| | | | | | | | | | | 88.0 |
| | | | SG | NT | 42 | 29 | A-11 | 2.52 | 48.5 | 51.3, 66.5, |
| | | | | | | | | | | 70.1, 90.4 |
| R12 | RNA:Valine:DPPC: | t = 0 | | 3.4 | 54 | 39 | A-5 | 3.14 | 50.2 | 66.9 |
| | Arginine:NaCl | t = 2 week | ND | NT | 44 | 25 | A-1 | 2.28 | none | 69.9, 86.3 |
| | (25:50:18:5:2) | | SG | NT | 41 | 21 | A-1 | 1.76 | 56.1 | 70.2 |
| | | t = 1 month | ND | NT | 47 | 25 | A-1 | 2.18 | none | 69.2, 91.6 |
| | | | SG | NT | 39 | 20 | A-1 | 1.87 | 59.9 | 69.8 |
| | | t = 3 month | ND | NT | 40* | 23* | A-1 | 2.07 | none | 68.7, 96.9 |
| | | | SG | NT | 38* | 19* | A-1 | 1.75 | none | 70.2 |
| | | t = 6 month | ND | NT | 40 | 22 | A-1 | 2.11 | none | 66.6, 100.1 |
| | | | SG | | 40 | 16 | A-1 | 1.74 | none | 69.8, 100.2 |
| R13 | RNA:Valine:DPPC: | t = 0 | | No powder was collected at the end of the run | | | | | | |
| | Arginine:NaCl | | | | | | | | | |
| | (25:50:18:5:2) | | | | | | | | | |
| R14 | RNA:Isoleucine:DPPC: | t = 0 | | 1.8 | 60 | 48 | A-7 | 1.73 | 48.7 | 62.4 |
| | NaCl | t = 2 week | ND | NT | 41 | 28 | A-7 | 1.74 | 50.4 | 65.6, 88.2 |
| | (25:55:18:2) | | SG | NT | 43 | 27 | A-7 | 1.46 | 48.9 | 65.6, 87.2 |
| | | t = 1 month | ND | NT | 39 | 22 | A-7 | 1.54 | 48.8 | 64.6, 92.0 |
| | | | SG | NT | 40 | 27 | A-7 | 1.48 | 50.5 | 65.3, 92.9 |
| | | t = 6 month | ND | NT | 48 | 31 | A-7 | 1.457 | none | 63.4, 98.6 |
| | | | SG | NT | 49 | 32 | A-7 | 1.457 | none | 64.5, 96.0 |

*Capsules did not, or may not have, spun during ACI-3 testing.
Legend:
XRPD (X-Ray Powder Diffraction); gPSD₅₀ (Average geometric particle size in microns (d50)); TGA-120: Total weight loss or volatiles from heating a sample up to 120° C. at a heating ramp of 20° C./min.

### Example 5—Results for Spray Dried Powders with RNA

Table 6 presents results obtained for the spray dried powders with RNA.

TABLE 6

| | | | | | FPF | FPF | | TGA- | | | |
|---|---|---|---|---|---|---|---|---|---|---|---|
| | | | Atomization | gPSD₅₀ | <5.6 um | <3.4 um | | 120° C. | Low T1 | Low T2 | Yield |
| Study | Batch # | Formulation | Rate (g/min) | (um) | (%) | (%) | XRPD | (%) | (° C.) | (° C.) | (%) |
| R1 | 449043 | RNA:Arginine:DPPC:NaCl | 40 | No powder was collected at the end of the run | | | | | | | 0 |
| | | (10:70:18:2) | | | | | | | | | |
| R2 | 483016 | RNA:Trehalose:DPPC:Tris: | 40 | 3.3 | 61 | 46 | NT | NT | NT | NT | 4.5 |
| | | EDTA (10:53:18:15:4) | | | | | | | | | |
| R3 | 483022 | RNA:Trehalose:DPPC:Tris: | 35 | 2.0 | 61 | 50 | A-1 | 2.67 | 42.4 | 52.8, 62.4, | 30.2 |
| | | EDTA (10:52:18:15:5) | | | | | | | | 73.6, 80.2 | |
| R4 | 483028 | RNA:Leucine:DPPC: | 30 | 2.5 | 75 | 56 | A-2 | 0.26 | none | 72.2, 78.3, | 26.4 |
| | | NaCl (1:79:18:2) | | | | | | | | 84.5, 89.3 | |
| R5 | 483045 | RNA:Trehalose:DPPC:NaCl | 25 | 3.8 | 71 | 55 | A-1 | 2.93 | 51.85 | 59.0, 69.2 | 15.8 |
| | | (1:79:18:2) | | | | | | | | | |
| R6 | 483071 | RNA: Trehalose:DPPC:PEI: | 30 | 3.3 | 77 | 63 | A-1 | 3.22 | 50.01 | 56.3, 65.3 | 23.8 |
| | | NaCl (1:74:18:5:2) | | | | | | | | | |
| R7 | 483072 | RNA:Trehalose:DPPC:PEI: | 30 | 5.0 | 56 | 36 | A-1 | 3.89 | 49.12 | 55.1, 66.2 | 12.8 |
| | | NaCl (1:74:18:5:2) | | | | | | | | | |
| R8 | 483084 | RNA:Trehalose:DPPC:PEI: | Run was not performed because the addition of PEI caused the RNA to crash out of solution | | | | | | | | |
| | | NaCl (10:65:18:5:2) | | | | | | | | | |
| R9 | 483085 | RNA: Trehalose:DPPC:NaCl | 30 | 2.7 | 68 | 54 | A-1 | 3.50 | 50.71 | 68.28 | 27.1 |
| | | (25:55:18:2) | | | | | | | | | |
| R10 | 483107 | RNA:Trehalose:DPPC:PEI: | 30 | 4.9 | 54 | 38 | A-1 | 3.96 | 49.8 | 54.8, 66.4 | 15.2 |
| | | NaCl (1:74:18:5:2) | | | | | | | | | |
| R11 | 483109 | RNA:Isoleucine:DPPC:NaCl | 30 | 2.2 | 49 | 34 | A-1 | 3.55 | 25.6 | 50.4, 62.9 | 16.7 |
| | | (50:30:18:2) | | | | | | | | | |
| R12 | 483119 | RNA:Valine:DPPC: | 30 | 3.4 | 54 | 39 | A-3 | 3.14 | 50.2 | 66.9 | 22.3 |
| | | Arginine:NaCl (25:50:18:5:2) | | | | | | | | | |
| R13 | 483120 | RNA:Valine:DPPC: | 30 | No powder was collected at the end of the run | | | | | | | 0 |
| | | Arginine:NaCl (25:50:18:5:2) | | | | | | | | | |
| R14 | 483132 | RNA:Isoleucine:DPPC:NaCl | 30 | 1.8 | 60 | 48 | A-4 | 1.73 | 48.7 | 62.4 | 13.3 |
| | | (25:55:18:2) | | | | | | | | | |

Legend:
XRPD (X-Ray Powder Diffraction); gPSD₅₀ (Average geometric particle size in microns (d50)); TGA-120: Total weight loss or volatiles from heating a sample up to 120° C. at a heating ramp of 20° C./min.

### Example 6—Discussion and Observations of Data and Results from Example 1-5

Spray-drying results above indicate light/dispersible powders can be formed.
High load achievable with sufficient FPF<5.6 μm and low residual solvent.
RNA powder can be formulated with positively charged cationic excipients.
R1 and R2 had to be aborted because of powder build up on the filter bag.
The powder stuck to the filter bag in R1 and R2 rapidly absorbed moisture once exposed to the atmosphere.
For R3 the filter bag was placed in a room at <15% RH for 2 days before use.
In both R2 and R3 silica gel is required in order to maintain the FPF observed at t=0.
The high mRNA and salt content is most likely why the FPF decreases without a desiccant.t
The presence of silica gel reduces the amount of moisture in the product over time.
The reduced atomization gas rate in R5 caused a thin layer of moisture to build up in the pipe at the exit of the drying cylinder.
This may be the reason for the large decrease in yield between R4 and R5.
The layer of moisture may also be the reason for the increase in the residual moisture detected by TGA.
In both R4 and R5 silica gel is required in order to maintain the FPF observed at t=0.
The lower mRNA and salt content increases the stability of the product without a desiccant present.
In R7 the filter bag was pulsed twice every 20 minutes to keep the pressure of the system from increasing.
In R7 the smaller particles were not collected by the cyclone resulting in a higher gPSD(50) and a smaller FPF.
If the size distribution was increased overall then the cyclone would have a more comparable yield.
The solid state data for R6 and R7 are very similar.
Using the cyclone and filter bag house mainly effects size distribution and yield rather than solid state properties.
The addition of a cationic excipient into the aqueous phase for Formulation 8 caused the mRNA to precipitate out of solution at 10% mRNA.
This did not occur at 1% mRNA (R6) or in the absence of the cationic excipient (R5 and R9).
In R7 the system was pulsed twice every 20 minutes to keep the system pressure from increasing (this was done once every 10 minutes in R10).
In R10 the In R7 and R10 the smaller particles were not collected by the cyclone resulting in a higher gPSD(50) and a smaller FPF.
If the size distribution was increased overall then the cyclone would have a more comparable yield.
R6, R7, and R10 have similar solid state data.
Using the cyclone and filter bag house mainly effects the size distribution and yield rather than solid state properties.
The density of the powder from R11 was noticeably denser than in R5 and R9.
At similar processing conditions, the increase in yeast RNA load from 25 to 50% resulted in a decrease in FPF.
In R12 an alternative positively charged excipient was used after the first cationic excipient caused the yeast RNA to precipitate. This may not be an issue for the GT mRNA as the yeast RNA is high MW and variable in distribution.
R12 had a bimodal gPSD, potentially due to crystallization of one of the amino acid excipients.
In R13 the new filter bag's diameter was too large and the bag fell off during the run. Contacted Core Filtration to resize the new bag Will increase the diameter of the lip or have a snap band added to the top of the bag.
RNA has been successfully loaded from 1% to 50%.
Yeast RNA adequately soluble in water & compatible with excipients.
Various Excipients tested including an array of amino acids and sugars.
Powders produced with cationic excipients for optimized drug uptake.
Fine Particle Fraction (FPF<5.6 μm) of up to 60-80% produced.
Testing physical stability of amorphous phase at accelerated storage conditions ongoing.
Chemical stability (being assessed at 1-6 months at 40° C./75% RH).
Desiccant and non-desiccant configurations being used for long term stability testing.

### Example 7-Studies with Spray Dried Powder Formulations for the Delivery of RNA and mRNA

Objective—The objective of this study was to investigate a variety of parameters and formulations in studies referred to herein as studies R16-R43, using materials and procedures similar to those described in Examples 1-5 in order to spray dry an mRNA containing powder with suitable chemical, physical and aerosol properties for delivery using a dry powder inhaler system and particularly for use in the ARCUS® platform. In some batches, yeast RNA was used as a placeholder for mRNA.

The formulations certain dry powders tested are listed in Table 7.

TABLE 7

| No. | Formulation |
|---|---|
| R16 | RNA:Valine:DPPC:NaCl (25:55:18:2) |
| R17 | RNA:Leucine:DPPC:NaCl (25:55:18:2) |
| R18 | RNA:Leucine:DPPC:Arginine:NaCl (25:50:18:5:2) |
| R22 | RNA:Lactose:DPPC:NaCl (25:55:18:2) |
| R23 | RNA:SD-30:DPPC:NaCl (25:55:18:2) |
| R24 | RNA:Leucine:DPPC:Arginine:NaCl (25:50:18:5:2) |
| R25 | RNA:Trehalose:DPPC:NaCl (25:55:18:2) |
| R26 | RNA:Valine:DPPC:Arginine:NaCl (25:50:18:5:2) |
| R27 | RNA:Trehalose:DPPC:NaCl (1:79:18:2) |
| R28 | RNA:Trehalose:DPPC:NaCl (1:79:18:2) |
| R29 | Uncapped mRNA:Trehalose:DPPC:NaCl (1:79:18:2) |
| R30 | RNA:Trehalose:DPPC:NaCl (25:55:18:2) |
| R32 | RNA:Trehalose:DPPC:NaCl (25:55:18:2) |
| R34 | RNA:Leucine:DPPC:NaCl (25:55:18:2) |
| R35 | Uncapped mRNA:Leucine:DPPC:NaCl (1:79:18:2) |
| R36 | RNA:Lactose:DPPC:NaCl (25:55:18:2) |
| R37 | Uncapped mRNA:Lactose:DPPC:NaCl (1:79:18:2) |
| R39 | RNA:Leucine:DPPC:NaCl (1:79:18:2) |
| R40 | RNA:Lactose:DPPC:NaCl (1:79:18:2) |
| R42 | RNA:Lactose:DPPC:NaCl (1:79:18:2) |
| R43 | RNA:Lactose:DPPC:NaCl (25:55:18:2) |

### Materials and Methods—

Table 8 provides lists the materials used in the production of spray dry powders of formulas studied in this Example 7 for the delivery of RNA and mRNA.

TABLE 8

| Chemical | Abbreviation | Manufacturer |
|---|---|---|
| Yeast ribonucleic acid | RNA | Roche Diagnostics |
| | | GmbH |
| Uncapped messenger | Uncapped | Georgia Tech |
| ribonucleic acid | mRNA | |
| Dipalmitoylphosphatidylcholine | DPPC | Lipoid |
| Sodium chloride | NaCl | BDH |
| Trehalose dihydrate | Trehalose | Sigma Aldrich |
| α-Lactose monohydrate | Lactose | Sigma Aldrich |
| Stabilite ® SD30 polyglycitol | SD-30 | Ingredion |
| powder | | |
| Arginine | Arg | Amresco Life |
| | | Sciences |
| Isoleucine | Ile | Sigma Aldrich |
| Leucine | Leu | Sigma Aldrich |
| Valine | Val | Sigma Aldrich |
| Tris(hydroxymethyl)aminomethane | Tris | G Biosciences |
| Ethylenediaminetetraacetic acid | EDTA | BDH |
| Polyethlyenimine | PEI | Aldrich Chemistry |

Table 9 lists the abbreviations used in this Example 7.

TABLE 9
Abbreviations used in Example 7

| Abbreviation | Definitions |
|---|---|
| gPSD₅₀ | Average geometric particle size |
| FPF | Fine particle fraction |
| FPF <5.6 μm | Particles with an aerodynamic diameter less than 5.6 μm |
| FPF <3.4 μm | Particles with an aerodynamic diameter less than 3.4 μm |
| XRPD | X-ray powder diffraction |
| TGA | Thermal gravimetric analysis |
| TGA-120° C. | Total weight loss from heating a sample up to 120° C. |
| DSC | Differential Scanning Calorimetry |
| SG | 1 gram of silica gel |
| ND | No desiccant |
| NT | Not taken |
| FBH | Filter bag house |
| FB | Filter bag |
| A-1 | Peaks due to excipients at 7, 14.5, 18, 19.5, 21, 23, 26, 29.5, 32.5, 35, and 37.5 (2Θ) |
| A-2 | Peaks due to excipients at 6, 12, 19, 21, 24, 30, and 32.5 (2Θ) |
| A-3 | Amorphous, Peak at 21 (2Θ) |
| A-4 | Peaks due to excipients at 6, 12, 19, 21, 24, 30, 32.5, 36, and 38.5 (2Θ) |
| A-5 | Peaks due to excipients at 7, 14.5, 18, 19.5, 21, 23, 23.5, 26, 29.5, 32.5, and 37.5 (2Θ) |
| A-6 | Peaks due to excipients at 6, 12, 19, 21.5, 22.5, 24.5, 30.5, 31.5, 32.5, and 33.5 (2Θ) |
| A-7 | Peaks due to excipients at 6.5, 12.5, 18.5, 19, 20, 21, 22, 25, 32.5, 37, and 38.5 (2Θ) |
| A-8 | Peaks due to excipients at 21, 28, 29.5, and 35.5 (2Θ) |
| A-9 | Peaks due to excipients at 21.5, 28, 29.5, 31, and 35.5 (2Θ) |
| A-10 | Peaks due to excipients at 18, 20.5, 21, 28.5, 29.5, 31.5, and 35.5 (2Θ) |
| A-11 | Peaks due to excipients at 6.5, 12.5, 19, 21, 25, and 32.5 (2Θ) |
| A-12 | Amorphous, Peak at 6, 21 (2Θ) |
| A-13 | Amorphous, Peak at 7.5, 21 (2Θ) |

### Equipment Setup and Process Parameters

The spray dryer used for runs listed in Table 10 is comprised of a size 1 spray dryer (GEA, NIRO, Dusseldorf, Germany) and was equipped with a single-bag product filter bag-house. The product filter bag used in runs listed in Table 10, except R26, was model number P034582-016-210 (Donaldson Filtration Solutions, Bloomington, MN) and is made of polyester with PTFE bag (5.87″ diameter×37.50″ length) and has a flat width of 9.23″. The filter bag used in R26 was model number 18-9088 (Franklin Products, Haw River, NC) and is made of PTFE (9.313″ flat×37.5″ length). The air cap used for this evaluation was model number 67147 (Spraying Systems Co., Wheaton, IL) and the fluid cap used for this evaluation was model number 2850 (Spraying Systems Co., Wheaton, IL). Table 10 lists the target process parameter values used for the production of powders containing RNA.

TABLE 10
Study Formulations R1-R14

| Parameter | R1 | R2 | R3 | R4 | R5 | R6 | R7 | R8 | R9 | R10 | R11 | R12 | R13 | R14 |
|---|---|---|---|---|---|---|---|---|---|---|---|---|---|---|
| Inlet temperature (° C.) | 67 | 67 | 67 | 67 | 67 | 67 | 67 | N/A | 67 | 67 | 67 | 67 | 67 | 67 |
| Outlet temperature | 40 | 40 | 40 | 40 | 40 | 40 | 40 | N/A | 40 | 40 | 40 | 40 | 40 | 40 |
| (° C.) | | | | | | | | | | | | | | |
| Drying gas flow | 125 | 125 | 125 | 125 | 125 | 125 | 125 | N/A | 125 | 125 | 125 | 125 | 125 | 125 |
| rate (kg/hr) | | | | | | | | | | | | | | |
| Atomization gas | 40 | 40 | 35 | 30 | 25 | 30 | 30 | N/A | 30 | 30 | 30 | 30 | 30 | 30 |
| flow rate (kg/hr) | | | | | | | | | | | | | | |
| Aqueous flow rate | 15 | 15 | 15 | 15 | 15 | 15 | 15 | N/A | 15 | 15 | 15 | 15 | 15 | 15 |
| (mL/min) | | | | | | | | | | | | | | |
| Organic flow rate | 15 | 15 | 15 | 15 | 15 | 15 | 15 | N/A | 15 | 15 | 15 | 15 | 15 | 15 |
| (mL/min) | | | | | | | | | | | | | | |
| Liquid Skid | 60 | 60 | 60 | 60 | 60 | 60 | 60 | 60 | 60 | 60 | 60 | 60 | 60 | 60 |
| Circulating Bath | | | | | | | | | | | | | | |
| Temperature (° C.) | | | | | | | | | | | | | | |
| Atomization Tower | 60 | 60 | 60 | 60 | 60 | 60 | 60 | 60 | 60 | 60 | 60 | 60 | 60 | 60 |
| Circulating Bath | | | | | | | | | | | | | | |
| Temperature (° C.) | | | | | | | | | | | | | | |
| System pressure (″WC) | −2.0 | −2.0 | −2.0 | −2.0 | −2.0 | −2.0 | −2.0 | N/A | −2.0 | −2.0 | −2.0 | −2.0 | −2.0 | −2.0 |
| Filter bag pulse | 30 | 30 | 30 | 30 | 30 | 30 | 1200 | N/A | 30 | 600 | 30 | 30 | 30 | 30 |
| time (sec) | | | | | | | (twice) | | | | | | | |
| Pulse tank | 70 | 70 | 80 | 70 | 70 | 70 | 70 | N/A | 70 | 70 | 70 | 70 | 70 | 70 |
| pressure (psi) | | | | | | | | | | | | | | |
| Baghouse purge | 20 | 20 | 20 | 20 | 20 | 20 | N/A | N/A | 20 | 20 | 20 | 20 | 20 | 20 |
| rate (scfh) | | | | | | | | | | | | | | |
| Total solid | 4 | 4 | 4 | 4 | 4 | 4 | 4 | N/A | 4 | 4 | 4 | 4 | 4 | 4 |
| concentration (g/L) | | | | | | | | | | | | | | |
| Product | FBH | FBH | FBH | FBH | FBH | FBH | Cyclone | N/A | FBH | Small | FBH | FBH | FBH | FBH |
| collection method | | | | | | | | | | Cyclone | | | | |

TABLE 10
(Continued) Study Formulations R16-18, R22-R26, R30, R32, R34, R36.

| Parameter | R16 | R17 | R18 | R22 | R23 | R24 | R25 | R26 | R30 | R32 | R34 | R36 |
|---|---|---|---|---|---|---|---|---|---|---|---|---|
| Inlet temperature (° C.) | 67 | 67 | 67 | 67 | 67 | 67 | 67 | 67 | 91 | 91 | 91 | 91 |
| Outlet temperature | 40 | 40 | 40 | 40 | 40 | 40 | 40 | 40 | 60 | 60 | 60 | 60 |
| (° C.) | | | | | | | | | | | | |
| Drying gas flow rate | 125 | 125 | 125 | 125 | 125 | 125 | 125 | 125 | 125 | 125 | 125 | 125 |
| (kg/hr) | | | | | | | | | | | | |
| Atomization gas flow | 30 | 30 | 30 | 30 | 30 | 30 | 30 | 30 | 30 | 40 | 30 | 30 |
| rate (kg/hr) | | | | | | | | | | | | |
| Aqueous flow rate | 15 | 15 | 15 | 15 | 15 | 15 | 15 | 15 | 15 | 15 | 15 | 15 |
| (mL/min) | | | | | | | | | | | | |
| Organic flow rate | 15 | 15 | 15 | 15 | 15 | 15 | 15 | 15 | 15 | 15 | 15 | 15 |
| (mL/min) | | | | | | | | | | | | |
| Liquid Skid | 60 | 60 | Off | 60 | 60 | 60 | 60 | 60 | 60 | 60 | 60 | 60 |
| Circulating Bath | | | | | | | | | | | | |
| Temperature (° C.) | | | | | | | | | | | | |
| Atomization Tower | 60 | 60 | Off | 60 | 60 | 60 | 60 | 60 | 60 | 60 | 60 | 60 |
| Circulating Bath | | | | | | | | | | | | |
| Temperature (° C.) | | | | | | | | | | | | |
| System pressure (″WC) | −2.0 | −2.0 | −2.0 | −2.0 | −2.0 | −2.0 | −2.0 | −2.0 | −2.0 | −2.0 | −2.0 | −2.0 |
| Filter bag pulse time | 30 | 30 | 30 | 30 | 30 | 30 | 30 | 30 | 30 | 30 | 30 | 30 |
| (sec) | | | | | | | | | | | | |
| Pulse tank pressure | 70 | 70 | 70 | 70 | 70 | 70 | 70 | 70 | 70 | 70 | 70 | 70 |
| (psi) | | | | | | | | | | | | |
| Baghouse purge rate | 20 | 20 | 20 | 20 | 20 | 20 | 20 | 20 | 20 | 20 | 20 | 20 |
| (scfh) | | | | | | | | | | | | |
| Total solid | 4 | 4 | 4 | 4 | 4 | 4 | 4 | 4 | 4 | 4 | 4 | 4 |
| concentration (g/L) | | | | | | | | | | | | |
| Product collection | FBH | FBH | FBH | FBH | FBH | FBH | FBH | FBH w/ | FBH | FBH | FBH | FBH |
| method | | | | | | | | new FB | | | | |

Note for Table:
The cyclones in R7 and R10 were located between the pressure sensor at the exit of the spray dryer and the filter baghouse.

The spray dryer used for runs listed in Table 11 is a Buchi Mini Spray Dryer B-290 (Buchi, New Castle, DE). The Target process parameters for formulas R27-R29, R35, R37, R39 and R40 are listed in Table 11.

TABLE 11
Formulations 27-29, 35, 37, 39 and 40.

| Parameter | R27 | R28 | R29 | R35 | R37 | R39 | R40 |
|---|---|---|---|---|---|---|---|
| Inlet temperature (° C.) | 93 | 120 | 120 | 120 | 120 | 120 | 120 |
| Outlet temperature (° C.) | 40 | 60 | 60 | 60 | 60 | 60 | 60 |
| Drying gas flow rate (kg/hr) | 25 | 25 | 25 | 25 | 25 | 25 | 25 |
| Atomization gas flow rate (kg/hr) | 30 | 30 | 30 | 30 | 30 | 30 | 30 |
| Aqueous flow rate (mL/min) | 5 | 5 | 5 | 5 | 5 | 5 | 5 |
| Organic flow rate (mL/min) | 5 | 5 | 5 | 5 | 5 | 5 | 5 |
| System pressure (mbar) | −50 | −50 | −50 | −50 | −50 | −50 | −50 |
| Total solid concentration (g/L) | 2.95 | 2.95 | 2.95 | 2.95 | 2.95 | 2.95 | 2.95 |
| Product collection method | Cyclone | Cyclone | Cyclone | Cyclone | Cyclone | Cyclone | Cyclone |

Spraying RNA with Isoleucine, Valine, and Leucine—

R14, R16, and R17 were formulated with 25% RNA and had Isoleucine, Valine, and Leucine as the main excipient, respectively. The three amino acids had different particle size distributions. When Isoleucine was the main excipient the particle size distribution had a right tail and was slightly bimodal. When Valine was the main excipient the particle size distribution was bimodal. When Leucine was the main excipient the distribution was unimodal and had a long right tail. The FPF<5.6 μm for the three powders were similar and the FPF<3.4 μm were relatively similar. The FPF<5.6 μm was 60% in R14, 57% in R16, and 57% in R17 and the FPF<3.4 μm was 48% in R14, 42% in R16, and 45% in R17. All three of the powders produced were crystalline, although they did have different crystalline structures. The residual solvent content for the three powders produced is relatively low and ranges by 0.80%. The residual solvent content is 1.73% in R14, 2.53% in R16, and 2.35% in R17. Overall, the powders that were made with amino acids as the main excipient are relatively similar to each other.

Spray Drying Amino Acids with and without a Positively Charged Excipient—

R24 and R17 were formulated with 25% RNA, Leucine as the main excipient, and with and without Arginine, respectively. Studies R12 and R16 were formulated with 25% RNA, Valine as the main excipient, and with and without Arginine, respectively. The addition of Arginine caused the gPSD₅₀ to decrease by 0.3 μm, from 3.7 μm in R16 to 3.4 μm in R12, when Valine was the main excipient. It caused the gPSD₅₀ to increase by 0.4 μm, from 1.6 μm in R17 to 2.0 μm in R24, when Leucine was the main excipient. When Valine and Leucine were the main excipients the addition of Arginine caused the FPF<5.6 μm and the FPF<3.4 μm to decrease. The FPF<5.6 μm decreased by 3% when Valine was the main excipient and by 12% when Leucine was the main excipient. The FPF<3.4 μm decreased by 3% when Valine was the main excipient and by 9% when Leucine was the main excipient. The residual solvent content increased when Arginine was added to Valine and Leucine. When Valine was the main excipient the residual solvent content increased by 0.61% and when Leucine was the main excipient the residual solvent content increased by 0.15%. Powders with Leucine and Valine as the main excipient remained crystalline when Arginine was added, however, however the crystalline structure was changed. It is unlikely trends could be drawn from the four studies detailed here. Unlike changing a process parameter or increasing the amount of an excipient, adding a new excipient can change much of how the formulation interacts as it is mixed, atomized, and dried.

Spray Drying RNA with Trehalose, Lactose, and SD-30—

R9, R22, and R23 were formulated with 25% RNA and had Trehalose, Lactose, and SD-30 as the main excipient, respectively. Powders with Trehalose and Lactose both had normally distributed particle sizes. The particle size distribution for Trehalose has slightly longer tails than the distribution for Lactose. The particle size distribution for SD-30 was bimodal. The FPF<5.6 μm and the FPF<3.4 μm for the three powders were relatively similar. The FPF<5.6 μm was 68% in R9, 67% in R22, and 64% in R23 and the FPF<3.4 μm was 54% in R9, 49% in R22, and 53% in R23. All three of the powders produced were amorphous and had a single peak at 21 (2Θ). The residual solvent content for the three powders produced is relatively high and ranges by 0.43%. The residual solvent content is 3.50% in R9, 3.93% in R22, and 3.92% in R23. Overall the powders that were made with sugars as the main excipient are relatively similar to each other.

### Comparing Amino Acids and Sugars as the Main Excipient—

R14, R16, and R17 were formulated with 25% RNA and an amino acid as the main excipient. Studies R9, R22, and R23 were also formulated with 25% RNA, but had a sugar as the main excipient. The studies formulated with sugars as the main excipient had higher FPF <5.6 μm and higher FPF<3.4 μm than the studies formulated with amino acids as the main excipient. The FPF<5.6 μm for the powders formulated with a sugar ranged from 64% to 68% and the powders formulated with an amino acid ranged from 57% to 60%. The FPF <3.4 μm for the powders formulated with a sugar ranged from 49% to 54% and the powders formulated with an amino acid ranged from 42% to 48%. The powders with sugar also had higher residual solvent contents than the powders with amino acids. The powders with sugars had residual solvent contents that ranged from 3.50% to 3.93% and the powders with amino acids had residual solvent contents that ranged from 1.73% to 2.53%. The powders formulated with sugars were amorphous and the powders formulated with amino acids were crystalline.

### Increasing Outlet Temperature

R5, R25 and R30 were formulated with 25% RNA, Trehalose as the main excipient, and outlet temperatures of 40° C., 45° C., and 60° C., respectively. Studies R17 and R34 were formulated with 25% RNA, Leucine as the main excipient, and outlet temperatures of 40° C. and 60° C., respectively. Studies R22 and R36 were formulated with 25% RNA, Lactose as the main excipient, and outlet temperatures 40° C. and 60° C., respectively. In all three cases, increasing the outlet temperature of the spray dryer increased the gPSD₅₀ of the powder produced. This trend is not linear as a 5° C. increase in outlet temperature increased the gPSD₅₀ by 0.4 μm, from 2.7 μm in R9 to 3.1 μm in R25, whereas a 20° C. increase in outlet temperature increased the gPSD₅₀ by 3.3 μm, from 2.7 μm in R9 to 6.0 μm in R30. Increasing the outlet temperature also has more of an effect on powders with sugar as their main excipient compared to powders with amino acids as their main excipient. When Lactose was the main excipient a 20° C. increase in outlet temperature increased the gPSD₅₀ by 2.3 μm, from 3.7 μm in R22 to 6.0 μm in R36. When Leucine was the main excipient a 20° C. increase in outlet temperature increased the gPSD₅₀ by 0.3 μm, from 1.6 μm in R17 to 1.9 μm in R34.

The Powders with Leucine as the main excipient had a normally distributed particle size distribution at 40° C. Increasing the outlet temperature to 60° C. increased the width of the particle size distribution. Powders with sugar as the main excipient also had a normally distributed particle size distribution at 40° C. When the outlet temperature was increased to 60° C. the particle size distribution had a left tail and was slightly bimodal. This trend is not linear as a 5° C. increase in outlet temperature only increased the width of the particle size distribution.

Increasing the outlet temperature also increased the FPF<5.6 μm and the FPF<3.4 μm. When Trehalose was the main excipient increasing the outlet temperature by 5° C. slightly increased the FPF. It increased the FPF<5.6 μm from 68% to 69% and the FPF<3.4 μm from 54% to 57%. When the outlet temperature was increased from 40° C. to 60° C. the change in FPF was more pronounced. The FPF<5.6 μm increased from 68% to 78% and the FPF<3.4 μm increased from 54% to 68%. Increasing the outlet temperature has less of an impact on the FPF when Leucine and Lactose were the main excipients. When Leucine was the main excipient a 20° C. increase in outlet temperature increased the FPF<5.6 μm from 57% to 63% and the FPF<3.4 μm from 45% to 50%. When Lactose was the main excipient the FPF<5.6 μm increased from 67% to 68% and the FPF<3.4 μm increased from 49% to 56%.

Increasing the outlet temperature reduced the residual solvent content of the power. When Trehalose was the main excipient and the outlet temperature was increased by 20° C. the residual solvent content decreased by 0.51%, from 3.50% in R9 to 2.99% in R30. When Lactose was the main excipient the residual solvent content decreased by 1.40%, from 3.93% in R22 to 2.53% in R36. When Leucine was the main excipient the residual solvent content decreased by 0.34%, from 2.35% in R17 to 2.01% in R34. When Trehalose was the main excipient and the outlet temperature was increased by 5° C. the residual solvent content increased by 0.13%, from 3.50% in R9 to 3.63% in R25. This result is the opposite of what would be expected, since increasing the temperature of the spray dryer should increase the evaporation rates of the solvents in the spray dryer. It is likely the result of experimental error.

### Increasing Atomization Gas Rate—

R30 and R32 were formulated with 25% RNA, Trehalose as the main excipient, and atomization gas rates of 30 g/min and 40 g/min, respectively. Increasing the atomization gas rate decreased the gPSD₅₀ by 1.66 μm, from 5.99 μm in R30 to 4.33 μm in R32. The particle distribution had a left tail and was slightly bimodal when the atomization gas rate was 30 g/min. When the atomization gas rate was increased to 40 g/min the particle distribution became more bimodal. Increasing the atomization gas rate as also decreased the FPF of the powder. The FPF<5.6 μm decreased by 10%, from 78% in R30 to 68% in R32. The FPF <3.4 μm decreased by 8%, from 68% in R30 to 60% in R32. There was also a slight reduction in the residual solvent content when the atomization gas rate was increased. The residual solvent content decreased by 0.18%, from 2.99% in R30 to 2.81% in R32.

### Not Heating the Liquid Feed Streams

R18 and R24 were formulated with 25% RNA, Leucine as the main excipient, and Arginine as a positively charged excipient. In study R18, the liquid skid circulating bath and the atomization tower circulating bath were not turned on. When the organic and aqueous solution mixed in the static mixer a white powder precipitated out of solution. The white powder was likely Leucine or Arginine. In study R24, when the liquid skid circulating bath and the atomization tower circulating bath were set to 60° C., nothing precipitated out of solution. Some formulations will require the liquid feeds to be heated before they enter the static mixer.

### Comparing the Polyester Product Filter Bag and the PTFE Product Filter Bag

R12 and R26 were formulated with 25% RNA, Valine as the main excipient, and Arginine as a positively charged excipient. In study R12 a filter bag made of polyester felt with a PTFE membrane was used. This is the same filter bag that was used in studies R1-R25, R27-R28, R30-R34, and R36. In study R26 a filter bag made entirely out of PTFE was used. The powders produced in these studies had similar solid-state data. The residual solvent content only increased 0.1%, from 3.14% in R12 to 3.24% in R26. The data obtained from the DSC was also similar. The Low T1 was 50.21° C. in R12 and was 48.58° C. in R26. The Low T2° C. in R12 was 66.87° C. in R12 and 65.06 in R26. The aerosol data between the two powders is less consistent. The gPSD₅₀ increased by 1.2 μm, from 3.4 μm in R12 to 4.6 μm in R26. The particle size distribution is bimodal in both R12 and R26, however, in R12 the first peak slightly is larger and in R26 the second peak is slightly larger. This could be because more particles of a particular size adhere to the polyester filter bag. The FPF also increased from R12 to R26. The FPF<5.6 μm increased by 1%, from 54% in R12 to 55% in R26 and the FPF<3.4 μm increased by 7%, from 39% in R12 to 46% in R26.

With the current filter bag it normally takes about 30 minutes for powder to start accumulating in the IBC. Using the new filter bag it only took about 10 minutes for powder to start accumulating in the IBC. The yield was also significantly higher with the new filter bag. The yield was up 15.5%, from 22.3% in R12 to 37.8% in R26.

Comparing Yeast RNA to mRNA—

R29, R35, and R37 were formulated with 1% mRNA and had Trehalose, Leucine, and Lactose as the main excipient, respectively. R28, R39, and R40 were formulated with 1% RNA and had Trehalose, Leucine, and Lactose as the main excipient, respectively.

There were slight changes in the gPSD₅₀ when RNA was replaced with mRNA. When Trehalose was the main excipient the gPSD₅₀ increased by 0.1 μm, from 1.3 μm in R28 to 1.4 μm in R29. When Leucine was the main excipient the gPSD₅₀ decreased by 0.4 μm, from 1.7 μm in R39 to 1.3 μm in R35. When Lactose was the main excipient the gPSD₅₀ increased by 1.0 μm, from 2.2 μm in R40 to 1.2 μm in R37. The changes in the gPSD₅₀ are most likely due to variability in the flow rates of the aqueous and organic feeds. The flow rates, for both the aqueous and organic feeds, were set to 5 mL/min for all of the runs that were performed on the Buchi. During the runs, the flow rates frequently deviated from the set point by 0.5 mL/min for extended periods of time. This offset would cause a change in the gPSD₅₀.

The changes in the FPF<5.6 μm and FPF<3.4 μm can be correlated to the changes in the gPSD₅₀. When Trehalose was the main excipient and 1% RNA was used, the capsule did not spin when ACI-3 was performed. When Trehalose was the main excipient and 1% mRNA was used the capsule did spin when ACI-3 was performed. For that reason, it is better to use the 2 week stability data when comparing the FPF data for R28 and R29 because the capsule spun with RNA and with mRNA. Since it is on stability with and without a desiccant, the average FPF values will be used for R28 and R29. When Trehalose was the main excipient the FPF<5.6 μm decreased 2%, from 58% in R28 to 56% in R29, and the FPF<3.4 μm decreased 6%, from 49% in R28 to 43% in R29. When Leucine was the main excipient the FPF<5.6 μm increased 19%, from 60% in R39 to 79% in R35, and the FPF<3.4 μm increased 19%, from 50% in R39 to 69% in R35. When Lactose was the main excipient the FPF <5.6 μm increased 28%, from 37% in R40 to 65% in R37, and the FPF<3.4 μm increased 33%, from 24% in R40 to 57% in R37. When the change in gPSD₅₀ is correlated to the change in FPF<5.6 μm a linear trend is observed. The trend line has the equation (ΔFPF<5.6 μm)=−26.9*(gPSD₅₀)+3.4 and an R²=0.92. When the change in gPSD₅₀ is correlated to the change in FPF<3.4 μm a linear trend is also observed. The trend line has the equation (ΔFPF<3.4 μm)=−35.1*(gPSD₅₀)+0.1 and an R²=0.95. Using these trends, if there was no change in the gPSD₅₀ when RNA was replaced with mRNA, then a 3.4% increase in the FPF<5.6 μm and a 0.10% increase in the FPF<3.4 μm would be expected.

When RNA was replaced with mRNA powders produced with a sugar as the main excipient had an increase in the residual solvent content. When Trehalose was the main excipient the residual solvent content increased by 0.39%, from 2.21% in R28 to 2.60% in R29. When Lactose was the main excipient the residual solvent content increased by 0.13%, from 2.36% in R40 to 2.49% in R37. The increase in the residual solvent content was larger when Trehalose was the main excipient. This is because the gPSD₅₀ increased when RNA was replaced with mRNA and it decreased when Lactose was the main excipient. An increase in the gPSD₅₀ means that the ratio of total liquid flow rate to atomization gas rate is higher. A higher ratio of total liquid flow rate to atomization gas rate would lead to an increase in the residual solvent content. The residual solvent content increased when the gPSD₅₀ decreased, this means that powders produced with mRNA have higher residual solvent contents than powders produced with RNA. When Leucine was the main excipient the residual solvent content decreased by 0.038%, from 0.500% in R39 to 0.462% in R35. This change is small enough to be considered noise.

The crystalline structure of the powders produced was unchanged when RNA was replaced with mRNA for all three excipients. The DSC data was very similar when trehalose and lactose were used as the main excipient. When lactose was the main excipient the DSC data was very similar before 90° C., but there were slight deviations after that point. Based on this it is likely that powders produced with RNA would be similar to powders produced with mRNA.

### Particle Characterization and Stability Data

Table 13 provides particle characterization for the listed formulations as described above. Table 14 provides stability data and particle characterization from T=0 up to T=6 months for the formulations listed and described above.

TABLE 13

| | | gPSD₅₀ | FPF | FPF | | TGA-120° | Low T1 | Low T2 | |
|---|---|---|---|---|---|---|---|---|---|
| Study | Formulation | (um) | <5.6 um (%) | <3.4 um (%) | XRPD | C. (%) | (° C.) | (° C.) | Yield (%) |
| R16 | RNA:Valine:DPPC:NaCl | 3.7 | 57 | 42 | A-1 | 2.53 | 48.0 | 66.5, 70.5 | 22.9 |
| | (25:55:18:2) | | | | | | | | |
| R17 | RNA:Leucine:DPPC:NaCl | 1.6 | 57 | 45 | A-2 | 2.35 | 47.5 | 63.9 | 10.8 |
| | (25:55:18:2) | | | | | | | | |
| R18 | RNA:Leucine:DPPC:Arginine:NaCl | Portion of formulation crashed out of solution in the static mixer because | | | | | | | 11.1 |
| | (25:50:18:5:2) | the heat exchangers was not used during the run. | | | | | | | |
| R22 | RNA:Lactose:DPPC:NaCl | 3.7 | 67 | 49 | A-3 | 3.93 | 47.9, 55.3 | 65.4 | 27.3 |
| | (25:55:18:2) | | | | | | | | |
| R23 | RNA:SD-30:DPPC:NaCl | 3.3 | 64 | 53 | A-3 | 3.92 | 46.1 | 71.6 | 32.9 |
| | (25:55:18:2) | | | | | | | | |
| R24 | RNA:Leucine:DPPC:Arginine:NaCl | 2.0 | 45 | 36 | A-4 | 2.50 | 49.4 | 66.6 | 24.3 |
| | (25:50:18:5:2) | | | | | | | | |
| R25 | RNA:Trehalose:DPPC:NaCl | 3.1 | 69 | 57 | A-3 | 3.63 | 45.4 | 66.1 | 29.6 |
| | (25:55:18:2) | | | | | | | | |
| R26 | RNA:Valine:DPPC:Arginine:NaCl | 4.6 | 55 | 46 | A-5 | 3.24 | 48.5 | 65.1 | 37.8 |
| | (25:50:18:5:2) | | | | | | | | |
| R27 | RNA:Trehalose:DPPC:NaCl | 1.5 | 56 | 22 | A-3 | 3.52 | 49.0 | 56.5, 64.3 | 40.9 |
| | (1:79:18:2) | | | | | | | | |
| R28 | RNA:Trehalose:DPPC:NaCl | 1.3 | 36 | 29 | A-3 | 2.21 | 36.8 | 69.8, 76.8 | 45.3 |
| | (1:79:18:2) | | | | | | | | |
| R29 | Uncapped | 1.4 | 60 | 48 | A-3 | 2.60 | 39.4 | 62.2, 70.4 | 53.4 |
| | mRNA:Trehalose:DPPC:NaCl | | | | | | | | |
| | (1:79:18:2) | | | | | | | | |
| R30 | RNA:Trehalose:DPPC:NaCl | 6.0 | 78 | 68 | A-3 | 2.99 | 42.8 | 68.9 | 38.8 |
| | (25:55:18:2) | | | | | | | | |
| R32 | RNA:Trehalose:DPPC:NaCl | 4.3 | 69 | 60 | A-3 | 2.81 | 39.1 | 69.6 | 24.8 |
| | (25:55:18:2) | | | | | | | | |
| R34 | RNA:Leucine:DPPC:NaCl | 1.9 | 63 | 50 | A-2 | 2.01 | 40.3 | 63.2 | 5.1 |
| | (25:55:18:2) | | | | | | | | |
| R35 | Uncapped | 1.3 | 79 | 69 | A-6 | 0.462 | 64.7 | 73.8, 80.4 | 40.5 |
| | mRNA:Leucine:DPPC:NaCl | | | | | | | | |
| | (1:79:18:2) | | | | | | | | |
| R36 | RNA:Lactose:DPPC:NaCl | 6.0 | 68 | 56 | A-3 | 2.53 | 40.5 | 72.6, 76.6 | 41.7 |
| | (25:55:18:2) | | | | | | | | |
| R37 | Uncapped | 1.2 | 65 | 57 | A-3 | 2.49 | 42.5 | 67.5, 72.3 | 42.6 |
| | mRNA:Lactose:DPPC:NaCl | | | | | | | | |
| | (1:79:18:2) | | | | | | | | |
| R39 | RNA:Leucine:DPPC:NaCl | 1.7 | 60 | 50 | A-6 | 0.500 | 61.8 | 77.3, 80.6 | 32.4 |
| | (1:79:18:2) | | | | | | | | |
| R40 | RNA:Lactose:DPPC:NaCl | 2.2 | 37 | 24 | A-3 | 2.36 | 38.3 | 69.6, 76.4 | 57.9 |
| | (1:79:18:2) | | | | | | | | |
| R42 | RNA:Lactose:DPPC:NaCl | 5.6 | 74 | 65 | A-3 | 3.16 | 41.7 | 70.4 | 30.3 |
| | (25:55:18:2) | | | | | | | | |
| R43 | RNA:Lactose:DPPC:NaCl | 3.03 | 76 | 62 | A-3 | 3.31 | 39.5 | 69.0 | 5.7 |
| | (25:55:18:2) | | | | | | | | |

Legend: XRPD (X-Ray Powder Diffraction); gPSD₅₀ (Average geometric particle size in microns (d50)); TGA-120: Total weight loss or volatiles from heating a sample up to 120° C. at a heating ramp of 20° C./min.

TABLE 14

| | | Conditions | | gPSD₅₀ | Size 00 FPF | Size 00 FPF | | TGA-120° |
|---|---|---|---|---|---|---|---|---|
| Study | Formulation | (40° C./75% RH) | | (um) | <5.6 um (%) | <3.4 um (%) | XRPD | C. (%) |
| R16 | RNA:Valine:DPPC:NaCl | t = 0 | | 3.7 | 57 | 42 | A-1 | 2.53 |
| | (25:55:18:2) | t = 2 week | ND | NT | 55 | 41 | A-1 | 1.91 |
| | | | SG | NT | 50 | 33 | A-1 | 1.72 |
| | | t = 1 month | ND | NT | 52 | 33 | A-1 | 1.78 |
| | | | SG | NT | 49 | 30 | A-1 | 1.60 |
| | | t = 3 month | ND | NT | 43 | 23 | A-1 | 1.55 |
| | | | SG | NT | 47 | 26 | A-1 | 1.48 |
| | | t = 6 month | ND | NT | 44 | 24 | A-1 | 1.56 |
| | | | SG | NT | 48 | 26 | A-1 | 1.50 |
| R17 | RNA:Leucine:DPPC:NaCl | t = 0 | | 1.6 | 57 | 45 | A-2 | 2.35 |
| | (25:55:18:2) | t = 2 week | ND | NT | 41 | 26 | A-2 | 1.95 |
| | | | SG | NT | 55 | 41 | A-2 | 1.61 |
| | | t = 1 month | ND | NT | 47 | 30 | A-2 | 1.70 |
| | | | SG | NT | 53 | 40 | A-2 | 1.60 |
| | | t = 5 month | ND | NT | 33 | 16 | A-2 | 1.61 |
| | | | SG | NT | 47 | 30 | A-2 | 1.48 |
| R18 | RNA:Leucine:DPPC:Arginine:NaCl | t = 0 | | Material crashed out in static mixer. | | | | |
| | (25:50:18:5:2) | | | | | | | |
| R22 | RNA:Lactose:DPPC:NaCl | t = 0 | | 3.7 | 67 | 49 | A-3 | 3.93 |
| | (25:55:18:2) | t = 2 week | ND | NT | 66 | 51 | A-3 | 3.21 |
| | | | SG | NT | 64 | 50 | A-3 | 2.85 |
| | | t = 1 month | ND | NT | 69 | 50 | A-3 | 3.11 |
| | | | SG | NT | 70 | 52 | A-3 | 2.73 |
| | | t = 3 month | ND | NT | 67 | 51 | A-3 | 3.067 |
| | | | SG | NT | 71 | 55 | A-3 | 2.596 |
| | | t = 6 month | ND | NT | 62 | 47 | A-13 | 3.074 |
| | | | SG | NT | 67 | 51 | A-3 | 2.626 |
| R23 | RNA:SD-30:DPPC:NaCl | t = 0 | | 3.3 | 64 | 53 | A-3 | 3.92 |
| | (25:55:18:2) | t = 2 week | ND | NT | 66 | 51 | A-3 | 3.21 |
| | | | SG | NT | 64 | 50 | A-3 | 2.85 |
| | | t = 1 month | ND | NT | 66 | 51 | A-3 | 3.14 |
| | | | SG | NT | 68 | 51 | A-3 | 2.62 |
| | | t = 3 month | ND | NT | 72 | 58 | A-3 | 2.99 |
| | | | SG | NT | 71 | 57 | A-3 | 2.62 |
| | | t = 6 month | ND | NT | 71 | 56 | A-13 | 2.77 |
| | | | SG | NT | 66 | 51 | A-3 | 2.53 |
| R24 | RNA:Leucine:DPPC:Arginine:NaCl | t = 0 | | 2.0 | 45 | 36 | A-4 | 2.50 |
| | (25:50:18:5:2) | t = 2 week | ND | NT | 53 | 38 | A-4 | 2.07 |
| | | | SG | NT | 59 | 46 | A-4 | 1.75 |
| | | t = 1 month | ND | NT | 57* | 41* | A-4 | 2.03 |
| | | | SG | NT | 59 | 44 | A-4 | 1.70 |
| | | t = 3 month | ND | NT | 58* | 41* | A-4 | 1.90 |
| | | | SG | NT | 60 | 46 | A-4 | 1.66 |
| | | t = 6 month | ND | NT | 56 | 38 | A-4 | 1.92 |
| | | | SG | NT | 62 | 45 | A-4 | 1.65 |
| R25 | RNA:Trehalose:DPPC:NaCl | T = 0 | | 3.1 | 69 | 57 | A-3 | 3.63 |
| | (25:55:18:2) | t = 2 week | ND | NT | 69 | 53 | A-3 | 3.09 |
| | | | SG | NT | 72 | 57 | A-3 | 2.69 |
| | | t = 1 month | ND | NT | 67 | 51 | A-3 | 3.29 |
| | | | SG | NT | 72 | 57 | A-3 | 2.57 |
| | | t = 3 month | ND | NT | 66 | 51 | A-3 | 3.32 |
| | | | SG | NT | 75 | 60 | A-3 | 2.79 |
| | | t = 6 month | ND | NT | 64 | 49 | A-3 | 3.13 |
| | | | SG | NT | 70 | 57 | A-3 | 2.83 |
| R26 | RNA:Valine:DPPC:Arginine:NaCl | t = 0 | | 4.6 | 55 | 46 | A-5 | 3.24 |
| | (25:50:18:5:2) | | | | | | | |
| R27 | RNA:Trehalose:DPPC:NaCl | t = 0 | | 1.5 | 56* | 22* | A-3 | 3.52 |
| | (1:79:18:2) | | | | | | | |
| R28 | RNA:Trehalose:DPPC:NaCl | t = 0 | | 1.3 | 36* | 29* | A-3 | 2.20 |
| | (1:79:18:2) | t = 1 month | ND | NT | 57 | 48 | A-3 | 2.47 |
| | | | SG | NT | 59 | 50 | A-3 | 2.32 |
| R29 | Uncapped | t = 0 | | 1.4 | 60 | 48 | A-3 | 2.60 |
| | mRNA:Trehalose:DPPC:NaCl | t = 1 month | ND | NT | 54 | 40 | A-3 | 2.67 |
| | (1:79:18:2) | | SG | NT | 57 | 45 | A-3 | 2.16 |
| | | t = 3 month | ND | NT | 60* | 50* | A-3 | 2.67 |
| | | | SG | NT | 54 | 41 | A-3 | 2.29 |
| R30 | RNA:Trehalose:DPPC:NaCl | t = 0 | | 6.0 | 78 | 68 | A-3 | 2.99 |
| | (25:55:18:2) | t = 2 week | ND | NT | 74 | 64 | A-3 | 3.12 |
| | | | SG | NT | 78 | 67 | A-3 | 2.62 |
| | | t = 1 month | ND | NT | 75 | 67 | A-3 | 3.03 |
| | | | SG | NT | 85 | 75 | A-3 | 2.51 |
| | | t = 3 month | ND | NT | 74 | 61 | A-3 | 3.18 |
| | | | SG | NT | 74 | 62 | A-3 | 2.57 |
| | | t = 6 month | ND | NT | 74 | 61 | A-3 | 3.09 |
| | | | SG | NT | 78 | 68 | A-3 | 2.61 |
| R32 | RNA:Trehalose:DPPC:NaCl | t = 0 | | 4.3 | 69 | 60 | A-3 | 2.81 |
| | (25:55:18:2) | t = 2 week | ND | NT | 71 | 60 | A-3 | 3.04 |
| | | | SG | NT | 70 | 58 | A-3 | 2.76 |
| | | t = 1 month | ND | NT | 71 | 60 | A-3 | 3.04 |
| | | | SG | NT | 70 | 58 | A-3 | 2.76 |
| | | t = 3 month | ND | NT | 88 | 72 | A-3 | 3.00 |
| | | | SG | NT | 82 | 73 | A-3 | 2.66 |
| | | t = 6 month | ND | NT | 80 | 63 | A-3 | 2.87 |
| | | | SG | NT | 78 | 65 | A-3 | 2.57 |
| R34 | RNA:Leucine:DPPC:NaCl | t = 0 | | 1.9 | 63* | 50* | A-2 | 2.01 |
| | (25:55:18:2) | t = 1 month | ND | NT | 53 | 33 | A-2 | 1.69 |
| | | | SG | NT | 56 | 37 | A-2 | 1.48 |
| R35 | Uncapped | t = 0 | | 1.3 | 79* | 69* | A-6 | 0.462 |
| | mRNA:Leucine:DPPC:NaCl | t = 2 week | ND | NT | 77 | 68 | A-6 | 0.444 |
| | (1:79:18:2) | | SG | NT | 81 | 74 | A-6 | 0.461 |
| | | t = 1 month | ND | NT | 55* | 46* | A-6 | 0.418 |
| | | | SG | NT | 77 | 66 | A-6 | 0.484 |
| | | t = 3 month | ND | NT | 77* | 66* | A-6 | 0.377 |
| | | | SG | NT | 81 | 72 | A-6 | 0.369 |
| R36 | RNA:Lactose:DPPC:NaCl | t = 0 | | 6.0 | 68* | 56* | A-3 | 2.53 |
| | (25:55:18:2) | t = 2 week | ND | NT | 69 | 54 | A-3 | 2.57 |
| | | | SG | NT | 70 | 56 | A-3 | 2.21 |
| | | t = 1 month | ND | NT | 74 | 63 | A-3 | 2.73 |
| | | | SG | NT | 73 | 60 | A-3 | 2.42 |
| | | t = 3 month | ND | NT | 73 | 60 | A-3 | 2.76 |
| | | | SG | NT | 72 | 59 | A-3 | 2.40 |
| | | t = 6 month | ND | NT | 82 | 60 | A-3 | 2.67 |
| | | | SG | NT | 79 | 59 | A-3 | 2.39 |
| R37 | Uncapped | t = 0 | | 1.2 | 65 | 57 | A-3 | 2.45 |
| | mRNA:Lactose:DPPC:NaCl | t = 1 month | ND | NT | 66 | 56 | A-3 | 2.30 |
| | (1:79:18:2) | | SG | NT | 68 | 61 | A-3 | 2.08 |
| R39 | RNA:Leucine:DPPC:NaCl | t = 0 | | 1.7 | 60* | 50* | A-6 | 0.500 |
| | (1:79:18:2) | t = 1 month | ND | NT | 69 | 57 | A-6 | 0.513 |
| | | | SG | NT | 69 | 56 | A-6 | 0.536 |
| | | t = 3.5 month | ND | NT | 71 | 59 | A-6 | 0.368 |
| | | | SG | NT | 70 | 57 | A-6 | 0.424 |
| R40 | RNA:Lactose:DPPC:NaCl | t = 0 | | 2.2 | 37 | 24 | A-3 | 2.36 |
| | (1:79:18:2) | t = 2 month | ND | NT | 49 | 34 | A-3 | 2.12 |
| | | | SG | NT | 42 | 28 | A-3 | 2.13 |
| R42 | RNA:Lactose:DPPC:NaCl | t = 0 | 5.6 | 74 | 65 | A-3 | 3.16 | 41.7 |
| | (25:55:18:2) | t = 2 week | ND | NT | 75 | 66 | A-3 | 2.69 |
| | | | SG | NT | 78 | 70 | A-3 | 2.58 |
| | | t = 1 month | ND | NT | 76 | 67 | A-3 | 2.89 |
| | | | SG | NT | 76 | 66 | A-3 | 2.63 |
| | | t = 3 month | ND | NT | 75 | 62 | A-3 | 2.87 |
| | | | SG | NT | 74 | 61 | A-3 | 2.22 |
| | | t = 6 month | ND | NT | 82 | 72 | A-3 | 2.70 |
| | | | SG | NT | 81 | 70 | A-3 | 2.49 |
| R43 | RNA:Lactose:DPPC:NaCl | t = 0 | 3.03 | 76 | 62 | A-3 | 3.31 | 39.5 |
| | (25:55:18:2) | | | | | | | |

| | | Conditions | | Low T1 | Low T2 |
|---|---|---|---|---|---|
| Study | Formulation | (40° C./75% RH) | | (° C.) | (° C.) |
| R16 | RNA:Valine:DPPC:NaCl | t = 0 | | 48.0 | 66.5, 70.5 |
| | (25:55:18:2) | t = 2 week | ND | none | 66.4, 70.5 |
| | | | SG | none | 67.5 |
| | | t = 1 month | ND | none | 66.2, 70.2 |
| | | | SG | none | 67.5, 71.5 |
| | | t = 3 month | ND | none | 64.8, 68.7 |
| | | | SG | none | 67.5 |
| | | t = 6 month | ND | none | 63.2, 66.8 |
| | | | SG | none | 65.8, 70.2 |
| R17 | RNA:Leucine:DPPC:NaCl | t = 0 | | 47.5 | 63.9 |
| | (25:55:18:2) | t = 2 week | ND | none | 64.7 |
| | | | SG | 51.2 | 64.8 |
| | | t = 1 month | ND | none | 64.7 |
| | | | SG | none | 64.8 |
| | | t = 5 month | ND | none | 63.4, 68.9 |
| | | | SG | none | 64.0, 69.5 |
| R18 | RNA:Leucine:DPPC:Arginine:NaCl | t = 0 | | Material crashed out in static mixer. | |
| | (25:50:18:5:2) | | | | |
| R22 | RNA:Lactose:DPPC:NaCl | t = 0 | | 47.9, 55.3 | 65.4 |
| | (25:55:18:2) | t = 2 week | ND | 43.8 | 71.1, 76.4 |
| | | | SG | 56.2 | 72.5, 80.6 |
| | | t = 1 month | ND | none | 71.5, 78.0 |
| | | | SG | 56.5 | 72.8, 83.3 |
| | | t = 3 month | ND | 44.25 | 71.12, 80.78 |
| | | | SG | 56.16 | 73.20, 85.90 |
| | | t = 6 month | ND | none | 63.16, 68.85, 77.75 |
| | | | SG | 56.04 | 72.64, 87.04 |
| R23 | RNA:SD-30:DPPC:NaCl | t = 0 | | 46.1 | 71.6 |
| | (25:55:18:2) | t = 2 week | ND | 43.8 | 71.1, 76.4 |
| | | | SG | 56.2 | 72.5, 80.6 |
| | | t = 1 month | ND | 58.9 | 74.3, 85.0 |
| | | | SG | 53.0 | 76.1, 83.4 |
| | | t = 3 month | ND | 43.5 | 71.12, 86.28 |
| | | | SG | 50.1 | 74.5, 86.1, 100.3 |
| | | t = 6 month | ND | 41.8 | 63.0, 66.2, |
| | | | | | 69.0, 88.0 |
| | | | SG | none | 73.0, 87.2 |
| R24 | RNA:Leucine:DPPC:Arginine:NaCl | t = 0 | | 49.4 | 66.6 |
| | (25:50:18:5:2) | t = 2 week | ND | none | 68.4, 91.5 |
| | | | SG | none | 68.4, 91.0 |
| | | t = 1 month | ND | none | 68.5, 92.7 |
| | | | SG | none | 68.9, 97.4 |
| | | t = 3 month | ND | none | 68.7, 97.3 |
| | | | SG | none | 69.0, 74.5, 98.2 |
| | | t = 6 month | ND | none | 68.1, 97.5 |
| | | | SG | none | 68.3, 73.8 |
| R25 | RNA:Trehalose:DPPC:NaCl | T = 0 | | 45.4 | 66.1 |
| | (25:55:18:2) | t = 2 week | ND | 44.5 | 69.2, 75.1 |
| | | | SG | 54.4 | 70.7, 78.7 |
| | | t = 1 month | ND | 45.6 | 70.7, 78.5 |
| | | | SG | 56.7 | 72.1, 82.1 |
| | | t = 3 month | ND | 40.6 | 68.7, 70.2, 76.6 |
| | | | SG | 53.3 | 70.2, 80.6 |
| | | t = 6 month | ND | 41.3 | 63.2, 67.5, |
| | | | | | 69.4, 77.9 |
| | | | SG | 53.5 | 70.0, 81.5 |
| R26 | RNA:Valine:DPPC:Arginine:NaCl | t = 0 | | 48.5 | 65.1 |
| | (25:50:18:5:2) | | | | |
| R27 | RNA:Trehalose:DPPC:NaCl | t = 0 | | 49.0 | 56.5, 64.3 |
| | (1:79:18:2) | | | | |
| R28 | RNA:Trehalose:DPPC:NaCl | t = 0 | | 36.8 | 69.8, 76.8 |
| | (1:79:18:2) | t = 1 month | ND | 54.2 | 67.0, 78.9 |
| | | | SG | 36.0, 53.8 | 64.1, 77.9 |
| R29 | Uncapped | t = 0 | | 39.4 | 62.2, 70.4 |
| | mRNA:Trehalose:DPPC:NaCl | t = 1 month | ND | 53.6 | 64.7, 75.7 |
| | (1:79:18:2) | | SG | 58.2 | 69.2, 79.1 |
| | | t = 3 month | ND | 51.5 | 62.4, 75.5 |
| | | | SG | 54.3 | 66.6, 78.9 |
| R30 | RNA:Trehalose:DPPC:NaCl | t = 0 | | 42.8 | 68.9 |
| | (25:55:18:2) | t = 2 week | ND | 39.0, 50.2 | 69.2, 78.1 |
| | | | SG | 39.3, 50.6 | 68.3, 75.2 |
| | | t = 1 month | ND | 53.4 | 69.6, 78.7 |
| | | | SG | 52.4 | 70.2, 81.2 |
| | | t = 3 month | ND | 41.4 | 69.4, 79.1 |
| | | | SG | 40.7, 53.2 | 70.0, 81.9 |
| | | t = 6 month | ND | 44.0 | 68.3, 79.5 |
| | | | SG | 54.2 | 70.0, 83.4 |
| R32 | RNA:Trehalose:DPPC:NaCl | t = 0 | | 39.1 | 69.6 |
| | (25:55:18:2) | t = 2 week | ND | 39.4, 54.0 | 68.7, 77.2 |
| | | | SG | 38.9, 51.2 | 69.0, 78.9 |
| | | t = 1 month | ND | 39.4, 54.0 | 68.7, 77.2 |
| | | | SG | 38.9, 51.2 | 69.0, 78.9 |
| | | t = 3 month | ND | none | 69.4, 79.8 |
| | | | SG | 53.0 | 69.2, 81.5 |
| | | t = 6 month | ND | 42.0, 54.9 | 68.9, 81.4 |
| | | | SG | 54.9 | 69.8, 84.0 |
| R34 | RNA:Leucine:DPPC:NaCl | t = 0 | | 40.3 | 63.2 |
| | (25:55:18:2) | t = 1 month | ND | 34.5 | 67.5 |
| | | | SG | 47.1 | 67.0 |
| R35 | Uncapped | t = 0 | | 64.7 | 73.8, 80.4 |
| | mRNA:Leucine:DPPC:NaCl | t = 2 week | ND | none | 73.7, 80.0 |
| | (1:79:18:2) | | SG | none | 73.9, 80.6 |
| | | t = 1 month | ND | none | 73.8, 80.2 |
| | | | SG | none | 73.6, 79.8 |
| | | t = 3 month | ND | 64.7 | 73.2, 79.5 |
| | | | SG | 62.4 | 73.6, 80.0 |
| R36 | RNA:Lactose:DPPC:NaCl | t = 0 | | 40.5 | 72.6, 76.6 |
| | (25:55:18:2) | t = 2 week | ND | 55.8 | 74.5, 85.9 |
| | | | SG | 55.4 | 73.6, 83.8 |
| | | t = 1 month | ND | 54.7 | 72.5, 83.6 |
| | | | SG | 54.7 | 73.0, 85.3 |
| | | t = 3 month | ND | 53.5 | 70.7, 83.4 |
| | | | SG | 52.4 | 72.3, 86.5 |
| | | t = 6 month | ND | 44.8, 63.6 | 70.0, 85.3 |
| | | | SG | 48.3, 56.1 | 72.3, 89.1 |
| R37 | Uncapped | t = 0 | | 42.5 | 67.5, 72.3 |
| | mRNA:Lactose:DPPC:NaCl | t = 1 month | ND | none | 67.9, 79.1, 149.0 |
| | (1:79:18:2) | | SG | none | 72.3, 82.3, 161.3 |
| R39 | RNA:Leucine:DPPC:NaCl | t = 0 | | 61.8 | 77.3, 80.6 |
| | (1:79:18:2) | t = 1 month | ND | 60.7 | 71.7, 78.9 |
| | | | SG | 61.0 | 72.3, 79.1 |
| | | t = 3.5 month | ND | 58.7 | 71.0, 78.1 |
| | | | SG | 61.8 | 72.2, 79.4 |
| R40 | RNA:Lactose:DPPC:NaCl | t = 0 | | 38.3 | 69.6, 76.4 |
| | (1:79:18:2) | t = 2 month | ND | 27.6, 37.0, 56.4 | 69.7, 83.1 |
| | | | SG | 28.9, 36.6, 56.7 | 73.0, 83.8 |
| R42 | RNA:Lactose:DPPC:NaCl | t = 0 | 5.6 | 70.4 | |
| | (25:55:18:2) | t = 2 week | ND | 53.8 | 72.5, 81.4 |
| | | | SG | 56.7 | 74.7, 84.8 |
| | | t = 1 month | ND | 40.7, 53.2 | 71.3, 80.6 |
| | | | SG | 52.0 | 72.1, 83.1 |
| | | t = 3 month | ND | none | 71.4, 82.4 |
| | | | SG | none | 72.2, 85.3 |
| | | t = 6 month | ND | 41.8 | 70.7, 84.2 |
| | | | SG | none | 72.6, 87.6 |
| R43 | RNA:Lactose:DPPC:NaCl | t = 0 | 3.03 | 69.0 | |
| | (25:55:18:2) | | | | |

*Capsules did not, or may not have, spun during ACI-3 testing.
Legend: XRPD (X-Ray Powder Diffraction); gPSD₅₀ (Average geometric particle size in microns (d50)); TGA-120: Total weight loss or volatiles from heating a sample up to 120° C. at a heating ramp of 20° C./min.

### Example 8—Particle Size Distributions of Spray Dried Powders with RNA and mRNA—

Table 15 provides particle size distributions for the spray dried powders of Formulations 1-14 and 17-40 described in Examples 1-7.

TABLE 15

| | gPSD₁₀ | gPSD₁₆ | gPSD₅₀ | gPSD₈₄ | gPSD₉₀ | gPSD₉₉ |
|---|---|---|---|---|---|---|
| Number | (um) | (um) | (um) | (um) | (um) | (um) |
| R1 | No powder was collected at the end of the run | | | | | |
| R2 | 0.8 | 1.1 | 3.3 | 9.5 | 12.1 | 23.9 |
| R3 | 0.7 | 0.8 | 2.0 | 5.1 | 6.7 | 16.0 |
| R4 | 0.9 | 1.2 | 2.5 | 5.2 | 7.0 | 25.0 |
| R5 | 0.9 | 1.2 | 3.8 | 9.1 | 11.1 | 19.8 |
| R6 | 0.8 | 1.0 | 3.3 | 8.6 | 10.5 | 20.9 |
| R7 | 1.5 | 2.1 | 5.0 | 8.9 | 10.3 | 17.0 |
| R8 | Run was not performed because the addition of | | | | | |
| | PEI caused the RNA to crash out of solution | | | | | |
| R9 | 0.9 | 1.2 | 2.7 | 5.9 | 7.7 | 17.5 |
| R10 | 1.7 | 2.2 | 4.9 | 9.2 | 10.8 | 17.9 |
| R11 | 0.8 | 0.9 | 2.2 | 5.3 | 7.4 | 24.9 |
| R12 | 0.8 | 1.0 | 3.4 | 13.3 | 17.3 | 35.3 |
| R13 | No powder was collected at the end of the run | | | | | |
| R14 | 0.7 | 0.8 | 1.8 | 4.7 | 6.7 | 16.7 |
| R16 (1) | 0.9 | 1.1 | 3.9 | 15.0 | 18.9 | 36.0 |
| R16 (2) | 1.0 | 1.4 | 5.5 | 15.8 | 19.1 | 34.4 |
| R16 (3) | 0.8 | 1.1 | 3.6 | 14.1 | 17.8 | 34.6 |
| R17 | 0.7 | 0.8 | 1.6 | 3.5 | 4.5 | 13.7 |
| R18 | Portion of formulation crashed out of solution in the static | | | | | |
| | mixer because the heat exchangers was not used during the run. | | | | | |
| R22 | 1.1 | 1.4 | 3.7 | 9.2 | 11.8 | 22.1 |
| R23 | 1.0 | 1.3 | 3.3 | 11.7 | 16.5 | 35.9 |
| R24 | 0.7 | 0.9 | 2.0 | 4.7 | 6.1 | 13.2 |
| R25 | 1.0 | 1.3 | 3.1 | 8.3 | 11.0 | 24.5 |
| R26 (1) | 1.0 | 1.3 | 4.8 | 13.8 | 17.2 | 35.2 |
| R26 (2) | 1.0 | 1.4 | 5.1 | 14.2 | 17.6 | 36.0 |
| R26 (3) | 0.9 | 1.1 | 3.7 | 11.9 | 15.2 | 30.8 |
| R27 (1) | 0.7 | 0.8 | 2.6 | 156.3 | 163.3 | 173.8 |
| R27 (2) | 0.7 | 0.8 | 1.5 | 2.9 | 3.5 | 6.3 |
| R27 (3) | 0.7 | 0.8 | 1.5 | 2.9 | 3.5 | 6.3 |
| R28 | 0.6 | 0.7 | 1.3 | 2.6 | 3.1 | 6.6 |
| R29 | 0.6 | 0.7 | 1.4 | 2.9 | 3.5 | 7.3 |
| R30 (1) | 1.1 | 1.5 | 6.0 | 14.8 | 17.9 | 33.1 |
| R30 (2) | 1.0 | 1.5 | 6.0 | 14.8 | 18.0 | 34.1 |
| R30 (3) | 1.1 | 1.6 | 6.5 | 16.3 | 20.4 | 130.1 |
| R32 (1) | 0.9 | 1.2 | 4.9 | 14.3 | 17.8 | 35.4 |
| R32 (2) | 0.8 | 1.1 | 4.0 | 13.9 | 17.7 | 33.8 |
| R32 (3) | 0.8 | 1.1 | 4.1 | 12.8 | 16.2 | 31.7 |
| R34 | 0.7 | 0.8 | 1.9 | 5.0 | 6.4 | 17.0 |
| R35 | 0.6 | 0.7 | 1.3 | 2.5 | 2.9 | 4.8 |
| R36 | 1.1 | 1.5 | 6.0 | 14.2 | 17.3 | 33.5 |
| R37 | 0.6 | 0.7 | 1.2 | 2.3 | 2.7 | 5.3 |
| R39 | 0.7 | 0.8 | 1.7 | 3.5 | 4.2 | 9.3 |
| R40 (1) | 0.7 | 0.9 | 2.2 | 5.4 | 6.7 | 11.9 |
| R40 (2) | 0.7 | 0.9 | 2.1 | 5.3 | 6.6 | 12.2 |
| R40 (3) | 0.8 | 0.9 | 2.2 | 5.6 | 7.0 | 14.5 |

### Example 9—Automatic Versus Manual Spray Dryer Runs

Table 16 shows results from Automatic Versus Manual Spray Dryer Runs.

TABLE 16

| | Yeast | gPSD₅₀ | Size 00 FPF | Size 00 FPF | | TGA-120° | Low T1 | Low T2 | |
|---|---|---|---|---|---|---|---|---|---|
| Number | RNA Load | (um) | <5.6 um (%) | <3.4 um (%) | XRPD | C. (%) | (° C.) | c(° C.) | SD |
| R14 | 25% | 1.79 | 60 | 48 | 6.5, 12.5, 18.5, | 1.728 | 48.72 | 42.41 | Manual |
| | | | | | 19, 20, 21, 22, | | | | |
| | | | | | 25, 32.5, 37, 38.5 | | | | |
| R15 | 25% | 3.58 | 57 | 68 | 6.5, 12.5, 18.5, | 2.686 | n.c. | 48.00, 63.05 | Auto |
| (same formula | | | | | 19, 20, 21, 23.5, | | | | |
| as used in R14) | | | | | 25, 32.5, 37, 38.5 | | | | |
| R16 | 25% | 3.74 | 57 | 42 | 7, 14.5, 18, 19.5, | 2.529 | 47.99 | 66.48, 70.49 | Manual |
| | | | | | 21, 23.5, 26, 29.5, | | | | |
| | | | | | 32.5, 35, 35.7 | | | | |
| R19 | 25% | 2.83 | 55 | 37 | 7, 14.5, 18, 19.5, | 2.141 | 47.90 | 67.75, 81.6 | Auto |
| (same formula | | | | | 21, 23.5, 26, 29.5, | | | | |
| as used in R16 | | | | | 32.5, 35, 35.7 | | | | |

Legend: XRPD (X-Ray Powder Diffraction); gPSD₅₀ (Average geometric particle size in microns (d50)); TGA-120: Total weight loss or volatiles from heating a sample up to 120° C. at a heating ramp of 20° C./min.
Notes for Table 16:
R15 retained more solvent than R14
This is because the outlet temperature probe for the automatic spray dryer needs to be calibrated (reads 4° C. higher than the manual)
R16 was run with a bypass and an outlet at 44° C.
The pipe diameter on the bypass was too small and the fan was not able to keep the system at −2.0 ″WC
Pulsed the powder off of the bag after the run

### Example 10—Additional Excipient Testing

Table 17 shows the results for additional excipient testing of Formulas 12, 16 and 17.

TABLE 17

| | Yeast | gPSD₅₀ | Size 00 FPF | Size 00 FPF | | TGA-120° | Low T1 | Low T2 |
|---|---|---|---|---|---|---|---|---|
| Number | RNA Load | (um) | <5.6 um (%) | <3.4 um (%) | XRPD | C. (%) | (° C.) | c(° C.) |
| R16 | 25% | 3.74 | 57 | 42 | 7, 14.5, 18, | 2.529 | 47.99 | 66.48, 70.49 |
| | | | | | 19.5, 21, 23.5, | | | |
| | | | | | 26, 29.5, 32.5, | | | |
| | | | | | 35, 35.7 | | | |
| R12 | 25% | 3.4 | 54 | 39 | 7, 14.5, 18, | 3.142 | 50.21 | 66.87 |
| | | | | | 19.5, 21, 23, | | | |
| | | | | | 23.5, 26, 29.5, | | | |
| | | | | | 32.5, 37.5 | | | |
| R17 | 25% | 1.56 | 57 | 45 | 6, 12, 19, 21, | 2.351 | 47.54 | 63.92 |
| | | | | | 24, 30, 30.5 | | | |

Notes for Table 17-
Both R12 and R16 have a bimodal gPSD. R12 is crystalline at t = 0 and t = 1 month

### Example 11—Capped mRNA with Lactose

Table 18 shows the results of spray-drying capped mRNA with Lactose in a Buchi spray dryer and a Size 1 spray dryer.

TABLE 18

| | | Conditions | | gPSD₅₀ | Size 00 FPF | Size 00 FPF | | TGA-120° | Low T1 | Low T2 |
|---|---|---|---|---|---|---|---|---|---|---|
| Study | Formulation | (40° C./75% RH) | | (um) | <5.6 um (%) | <3.4 um (%) | XRPD | C. (%) | (° C.) | (° C.) |
| R44 | RNA:Lactose:DPPC:NaCl | t = 0 | | 2.31 | 44 | 33 | A-3 | 2.54 | 32.4, 50.3 | 64.1, 71.1, 163.0 |
| | (1:79:18:2) | t = 1 month | ND | NT | 36* | 24* | A-3 | 2.64 | 54.7 | 66.8, 78.5, |
| | | | | | | | | | | 152.3, 199.1 |
| | | | SG | NT | 10* | 6* | A-3 | 2.28 | 54.8 | 68.3, 80.9, |
| | | | | | | | | | | 148.9, 199.6 |
| | | t = 6 month | ND | NT | 43 | 27 | A-3 | 2.62 | 54.61 | 64.3, 79.4, |
| | | | | | | | | | | 140.6, 200.6 |
| | | | SG | NT | 43 | 28 | A-3 | 2.37 | 56.2 | 68.5, 83.8, |
| | | | | | | | | | | 157.7, 201.8 |
| R45 | RNA:Lactose:DPPC:NaCl | t = 0 | | 2.76 | 54 | 32 | A-3 | 2.35 | 37.9 | 69.8, 76.4 |
| | (10:70:18:2) | t = 1 month | ND | NT | 65 | 41 | A-3 | 2.27 | 55.5 | 69.7, 79.8 |
| | | | SG | NT | 66 | 42 | A-3 | 2.36 | 55.8 | 69.0, 80.1 |
| | | t = 6 month | ND | NT | 59 | 39 | A-3 | 2.37 | 54.6 | 67.8, 81.7 |
| | | | SG | NT | 67 | 45 | A-3 | 2.31 | 56.0 | 69.7, 83.5 |
| R46 | RNA:Lactose:DPPC:NaCl | t = 0 | | 5.80 | 79 | 69 | A-3 | 3.63 | 51.5 | 58., 66.5, 147.1 |
| | (1:79:18:2) | t = 2 week | ND | NT | 72 | 63 | A-3 | 2.52 | none | 64.9, 74.3, 153.9 |
| | | | SG | NT | 73 | 63 | A-3 | 2.35 | none | 70.0, 78.1, 164.0 |
| | | t = 1 month | ND | NT | 72 | 61 | A-3 | 2.61 | 56.1 | 64.8, 75.4, |
| | | | | | | | | | | 157.3, 200.5 |
| | | | SG | NT | 77 | 65 | A-3 | 2.12 | 60.7 | 71.1, 80.0, |
| | | | | | | | | | | 174.4, 201.9 |
| | | t = 3 month | ND | NT | 75 | 64 | A-3 | 2.69 | 59.7 | 64.4, 76.5, |
| | | | | | | | | | | 145.4, 197.7 |
| | | | SG | NT | 80 | 70 | A-3 | 2.25 | 62.2 | 71.7, 82.2, |
| | | | | | | | | | | 174.0, 201.3 |
| | | t = 6 month | ND | NT | 72 | 61 | A-3 | 2.78 | 62.9 | 72.5, 83.6, 171.3, |
| | | | | | | | | | | 187.0, 205.0 |
| | | | SG | NT | 79 | 70 | A-3 | 2.28 | 56.2 | 64.1, 76.4, 142.7, |
| | | | | | | | | | | 171.1, 205.8 |

Table 20 shows particle characterization results of capped mRNA with lactose formulations from R44-R46.

TABLE 20

| | | gPSD₅₀ | FPF | FPF | | TGA-120° | Low T1 | Low T2 | Yield |
|---|---|---|---|---|---|---|---|---|---|
| Study | Formulation | (um) | <5.6 um (%) | <3.4 um (%) | XRPD | C. (%) | (° C.) | (° C.) | (%) |
| R44 | Capped | 2.31 | 44 | 33 | A-3 | 2.54 | 32.4, 50.3 | 64.1, 71.1, | 71.4 |
| | RNA:Lactose:DPPC:NaCl | | | | | | | 163.0 | |
| | (1:79:18:2) | | | | | | | | |
| R45 | Capped | 2.76 | 54 | 32 | A-3 | 2.35 | 37.9 | 69.8, 76.4 | Not |
| | RNA:Lactose:DPPC:NaCl | | | | | | | | Recorded |
| | (10:70:18:2) | | | | | | | | |
| R46 | Capped | 5.80 | 79 | 69 | A-3 | 3.63 | 51.5 | 58., 66.5, | 35.8 |
| | RNA:Lactose:DPPC:NaCl | | | | | | | 147.1 | |
| | (1:79:18:2) | | | | | | | | |

Table 21 provides particle size distributions for the spray dried powders of R44-R46.

TABLE 21

| | gPSD₁₀ | gPSD₁₆ | gPSD₅₀ | gPSD₈₄ | gPSD₉₀ | gPSD₉₉ |
|---|---|---|---|---|---|---|
| Study | (um) | (um) | (um) | (um) | (um) | (um) |
| R44 | 0.8 | Not | 2.3 | Not | 5.8 | Not |
| | | Recorded | | Recorded | | Recorded |
| R45 | 0.8 | Not | 2.4 | Not | 7.7 | Not |
| | | Recorded | | Recorded | | Recorded |
| R46 (1) | 1.1 | 1.5 | 5.7 | 13.1 | 15.8 | 29.5 |
| R46 (2) | 1.1 | 1.5 | 5.9 | 13.6 | 16.4 | 29.8 |

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims. It will also be understood that none of the embodiments described herein are mutually exclusive and may be combined in various ways without departing from the scope of the invention encompassed by the appended claims.

1. A respirable, dry powder particle formulation for pulmonary delivery comprising:
i) at least about 1% RNA by weight of the particle;
ii) at least about 10% DPPC by weight of the particle;
iii) optionally, at least about 1% each of one or more of arginine, leucine, isoleucine, or valine by weight of the particle, wherein when two or more amino acids are selected, each amino acid has either the same or different weight percentage as the other selected amino acid(s);
iv) optionally, at least about 1% NaCl by weight of the particle;
v) optionally, at least about 10% Tris by weight of the particle;
vi) optionally, at least about 2% EDTA by weight of the particle;
vii) optionally, at least about 40% trehalose by weight of the particle;
ix) optionally, at least about 40% lactose by weight of the particle and
viii) optionally, at least about 2% PEI by weight of the particle;
wherein all components of the RNA dry powder amount to 100 weight percent.

2. The formulation of claim 1, wherein the powder has an FPF<5.6 μm of at least about 40% or more.

3. The formulation of claim 1, wherein the powder has an FPF<5.6 μm of at least about 45%, 50%, 55%, 60%, 65%, 70%, 75%, or more.

4. The formulation of claim 1, wherein the powder has an FPF<3.4 μm of at least about 30% or more.

5. The formulation of claim 1, wherein the powder has an FPF<3.4 μm of at least about 35%, 40%, 45%, 50%, 55%, 60%, 65%, 70%, or more.

6. The formulation of claim 1, wherein the gPSD₅₀ is about 1 micron to about 10 microns.

7. The formulation of claim 1, selected from Table 1:
TABLE 1

| Number | Formulation |
|---|---|
| 1 | RNA:Arginine:DPPC:NaCl (10:70:18:2) |
| 2 | RNA:Trehalose:DPPC:Tris:EDTA (10:53:18:15:4) |
| 3 | RNA:Trehalose:DPPC:Tris:EDTA (10:52:18:15:5) |
| 4 | RNA:Leucine:DPPC:NaCl (1:79:18:2) |
| 5 | RNA:Trehalose:DPPC:NaCl (1:79:18:2) |
| 6 | RNA:Trehalose:DPPC:PEI:NaCl (1:74:18:5:2) |
| 8 | RNA:Trehalose:DPPC:PEI:NaCl (10:65:18:5:2) |
| 9 | RNA:Trehalose:DPPC:NaCl (25:55:18:2) |
| 11 | RNA:Isoleucine:DPPC:NaCl (50:30:18:2) |
| 12 | RNA:Valine:DPPC:Arginine:NaCl (25:50:18:5:2) |
| 14 | RNA:Isoleucine:DPPC:NaCl (25:55:18:2) |
| 16 | RNA:Valine:DPPC:NaCl (25:55:18:2) |
| 17 | RNA:Leucine:DPPC:NaCl (25:55:18:2) |
| 18 | RNA:Leucine:DPPC:Arginine:NaCl (25:50:18:5:2) |
| 22 | RNA:Lactose:DPPC:NaCl (25:55:18:2) |
| 23 | RNA:SD-30:DPPC:NaCl (25:55:18:2) |
| 24 | RNA:Leucine:DPPC:Arginine:NaCl (25:50:18:5:2) |
| 25 | RNA:Trehalose:DPPC:NaCl (25:55:18:2) |
| 26 | RNA:Valine:DPPC:Arginine:NaCl (25:50:18:5:2) |
| 27 | RNA:Trehalose:DPPC:NaCl (1:79:18:2) |
| 29 | Uncapped mRNA:Trehalose:DPPC:NaCl (1:79:18:2) |
| 30 | RNA:Trehalose:DPPC:NaCl (25:55:18:2) |
| 34 | RNA:Leucine:DPPC:NaCl (25:55:18:2) |
| 35 | Uncapped mRNA:Leucine:DPPC:NaCl (1:79:18:2) |
| 36 | RNA:Lactose:DPPC:NaCl (25:55:18:2) |
| 37 | Uncapped mRNA:Lactose:DPPC:NaCl (1:79:18:2) |
| 39 | RNA:Leucine:DPPC:NaCl (1:79:18:2) |
| 40 | RNA:Lactose:DPPC:NaCl (1:79:18:2) |
| 44 | capped RNA:Lactose:DPPC:NaCl (1:79:18:2) |
| 45 | capped RNA:Lactose:DPPC:NaCl (10:70:18:2). |

8. A method of treating a patient suffering from a disease of the respiratory system, comprising administering at least one formulation of claim 1 by pulmonary delivery.

9. The method of claim 8, wherein the patient is an adult human or a pediatric human.

10. The method of claim 8, wherein the disease is selected from asthma, pulmonary arterial hypertension, chronic obstructive pulmonary disease (COPD), respiratory distress syndrome (RDS), chronic bronchitis, acute bronchitis, emphysema, cystic fibrosis, pneumonia, tuberculosis, lung cancer, acute respiratory distress syndrome (ARDS), pneumoconiosis, interstitial lung disease (ILD) (such as sarcoidosis, idiopathic pulmonary fibrosis, and autoimmune disease), pulmonary embolism, pleural effusion, and mesothelioma.

11. A pharmaceutical composition comprising the respirable, dry powder particle formulation for pulmonary delivery according to claim 1.

12. A respirable, dry powder particle formulation for pulmonary delivery comprising:
i) at least about 1% RNA polynucleotide by weight of the particle;
ii) at least about 10% DPPC by weight of the particle;
iii) optionally, at least about 1% each of one or more of arginine, leucine, isoleucine, or valine by weight of the particle, wherein when two or more amino acids are selected, each amino acid has either the same or different weight percentage as the other selected amino acid(s);
iv) optionally, at least about 1% NaCl by weight of the particle;
v) optionally, at least about 10% Tris by weight of the particle;
vi) optionally, at least about 2% EDTA by weight of the particle;
vii) optionally, at least about 40% trehalose by weight of the particle;
ix) optionally, at least about 40% lactose by weight of the particle and
viii) optionally, at least about 2% PEI by weight of the particle;
wherein all components of the RNA dry powder amount to 100 weight percent.

13. The method of claim 12, wherein the polynucleotide is a ribonucleic acids (RNA), short interfering RNA (siRNA), micro-RNA, deoxyribonucleic acid (DNA), threose nucleic acids (TNA), glycol nucleic acids (GNA), peptide nucleic acid (PNA), or a locked nucleic acid (LNA).

14. The method of claim 1, wherein the RNA is from about 10 to about 12,000 nucleotides in length.

15. The method of claim 12, wherein the polynucleotide is from about 10 to about 12,000 nucleotides in length.
